(19) United States
(12) Patent Application Publication
(10) Pub. No.: US 2012/0172298 A1
(43) Pub. Date: Jul. 5, 2012

(54) GLP-1 AND FGF21 COMBINATIONS FOR TREATMENT OF DIABETES TYPE 2

(75) Inventors: Birgitte Andersen, Malov (DK); Ann Maria Kruse Hansen, København Ø (DK); Bidda Charlotte Rolin, Nærum (DK)

(73) Assignee: Novo Nordisk A/S, Bagsvaerd (DK)

(21) Appl. No.: 13/375,360

(22) PCT Filed: Jun. 8, 2010
(86) PCT No.: PCT/EP2010/057986

§ 371 Date: Mar. 20, 2012

Related U.S. Application Data
(60) Provisional application No. 61/186,471, filed on Jun. 12, 2009.

(30) Foreign Application Priority Data
Jun. 11, 2009 (EP) 09162521.0

(51) Int. Cl. A61K 38/26; A61P 3/10

(52) U.S. Cl. 514/7.2

(57) ABSTRACT

The invention relates to the use of a Fibroblast Growth Factor 21 (FGF21) compound and a Glucagon-Like Peptide 1 (GLP-1) compound in combination for the preparation of a medicament for the treatment of diabetes, more in particular type 2 diabetes, as well as pharmaceutical compositions comprising certain FGF21 and GLP-1 compounds in combination, together with a pharmaceutically acceptable carrier. The combination has a significant effect on parameters of relevance for diabetes type 2, viz. on the viability of beta cells ex vivo in the presence of free fatty acids, on caspase activity of beta cells ex vivo (a measure of cell apoptosis), and a blood glucose lowering effect on db/db mice in vivo.

### FIELD OF THE INVENTION

The invention relates to the use of a Fibroblast Growth Factor 21 (FGF21) compound and a Glucagon-Like Peptide 1 (GLP-1) compound in combination for the preparation of a medicament for the treatment of diabetes, more in particular type 2 diabetes.

The invention also relates to pharmaceutical compositions comprising certain FGF21 and GLP-1 compounds in combination, together with a pharmaceutically acceptable carrier.

### BACKGROUND OF THE INVENTION

Fibroblast growth factors are polypeptides expressed in developing and adult tissues. They are involved in several physiological mechanisms including for example metabolic regulation and cellular differentiation. A whole family of more than twenty fibroblast growth factors exists (the FGF family). Three members of the FGF family including FGF19, FGF21, and FGF23 form a subfamily functioning as endocrine factors involved in metabolic regulation.

FGF21 is expressed preferentially in the liver and has been shown to exert hormone-like metabolic effects. The mature human FGF21 polypeptide has the sequence of amino acids 1-181 of SEQ ID NO:1.

FGF21 has been demonstrated to activate glucose uptake in mouse adipocytes, and to lower blood glucose and triglyceride levels when administered to diabetic rodents (Kharitonenkov et al., J. Clin. Invest. (2005), 115:1627-1635). The lowering effect of FGF21 on blood glucose and triglycerides has also been shown in diabetic monkeys. Based on these results FGF21 has been suggested as a pharmacological agent with the potential to treat i.a. diabetes.

GLP-1 is an incretin hormone produced by the endocrine cells of the intestine following ingestion of food. GLP-1 is a regulator of glucose metabolism, and the secretion of insulin from the beta cells of the islets of Langerhans in the pancreas. GLP-1 also causes insulin secretion in the diabetic state. The half-life in vivo of GLP-1 itself is, however, very short, thus, ways of prolonging the half-life of GLP-1 in vivo has attracted much attention.

WO 98/08871 discloses protracted GLP-1 analogues and derivatives based on human GLP-1(7-37) (amino acids 1-31 of SEQ ID NO:3) which have an extended half-life, including liraglutide, a GLP-1 derivative for once daily administration developed by Novo Nordisk A/S and expected to be marketed soon for the treatment of type 2 diabetes.

Exenatide is a commercial incretin mimetic for the treatment of diabetes mellitus type 2 which is manufactured and marketed by Amylin Pharmaceuticals and Eli Lilly & Co. Exenatide is based on exendin-4(7-45) (amino acids 1-39 of SEQ ID NO:4), a hormone found in the saliva of the Gila monster. It displays biological properties similar to human GLP-1. U.S. Pat. No. 5,424,286 relates i.a. to a method of stimulating insulin release in a mammal by administration of exendin-4(7-45) (SEQ ID NO:1 in the US patent).

WO 2009/020802 relates to the use of an FGF21 compound and a GLP-1 compound in the manufacture of a medicament for lowering body weight and for treatment of obesity based on an alleged synergistic effect. The combination is furthermore alleged—but only once, and very briefly, just in passing on page 7—to also result in “a synergistic effect on lower elevated blood glucose levels, and thus, a potential use in the treatment of diabetes”. However, the latter allegation is totally unsupported.

The present invention provides enablement and evidence of a significant effect on the treatment of diabetes type 2 by use of a combination of an FGF21 compound and a GLP-1 compound.

### SUMMARY OF THE INVENTION

The present invention relates to the use of an FGF21 compound and a GLP-1 compound in combination for the preparation of a medicament for the treatment of type 2 diabetes.

The present application provides a showing of surprising and unexpected significant effects of this combination, i.a., supported by studies in relation to the viability of beta cells ex vivo in the presence of free fatty acids; studies in relation to the caspase activity of beta cells ex vivo in the presence of free fatty acids (a measure of cell apoptosis); and/or studies showing a blood glucose lowering effect on db/db mice in vivo.

One of the compounds tested in combination with FGF21 is the novel compound N-epsilon-37-[2-(2-{2-[2-(2-{2-[(S)-4-carboxy-4-(15-carboxypentadecanoylamino)butyrylamino]ethoxy}ethoxy)-acetylamino]ethoxy}ethoxy)acetyl] [Aib8,22,35,Lys37]GLP-1-(7-37), in what follows “compound G4”.

The invention furthermore relates to:

a combination of an FGF21 compound and a GLP-1 compound for the treatment of type 2 diabetes;

a composition comprising an FGF21 compound and a GLP-1 compound, and a pharmaceutically acceptable carrier, wherein the GLP-1 compound:

i) comprises at least one of the following: DesaminoHis7, Aib8, Aib22, Arg26, Aib35, and/or Lys37;

ii) is a GLP-1 derivative comprising an albumin binding moiety which comprises at least one, preferably at least two, more preferably two, free carboxylic acid groups; or a pharmaceutically acceptable salt thereof;

iii) is a GLP-1 derivative comprising an albumin binding moiety that comprises an acyl radical of a dicarboxylic acid, preferably comprising a total of from 12 to 24 carbon atoms, such as C12, C14, C16, C18, C20, C22, or C24, most preferably C16, C18, or C20; wherein preferably a) the acyl radical is attached to the epsilon amino group of a lysine residue of the GLP-1 peptide via a linker; b) the linker comprises at least one OEG radical, and/or at least one Trx radical, and, optionally, additionally at least one Glu; and/or

iv) is selected from the compounds of claim 28, with the exception of compound G1; and/or the FGF21 compound:

a) comprises at least one of −1M, S71C, K56R, K59R, K69R, and/or K122R;

b) is an FGF21 derivative modified via the thiol group of a cysteine residue, preferably an internal cysteine residue, such as 71C;

c) is an FGF21 derivative comprising an albumin binding moiety;

d) is not a PEGylated FGF21 derivative;

e) is an FGF21 derivative comprising an albumin binding moiety which comprises at least one, preferably at least two, more preferably two, free carboxylic acid groups, or a pharmaceutically acceptable salt thereof;

f) is an FGF21 derivative comprising an albumin binding moiety that comprises an acyl radical, such as acyl of fatty acids or dicarboxylic acids, the acyl radical preferably comprising a total of from 12 to 24 carbon atoms, such as C12, C14, C16, C18, C20, C22, or C24, most preferably C18, or C20; or a pharmaceutically acceptable salt thereof; wherein preferably i) the acyl radical is attached to the amino group of the N-terminal amino acid residue of the FGF21 peptide, e.g. to the amino group of −1M, or to the thiol group of an internal cysteine residue of the FGF21 peptide, e.g. to the thiol group of 71C, via a linker; and preferably b) the linker comprises at least one OEG radical, and/or at least one Glu radical; and/or

g) is selected from the compounds of claim 51, with the exception of the polypeptide having SEQ ID NO: 1; as well as

methods of treating type 2 diabetes, improving the viability of beta cells, reducing apoptosis of beta cells, and lowering blood glucose, all methods comprising administering to a patient an effective amount of an FGF21 compound and a GLP-1 compound in combination.

In a further aspect, the present invention relates to a composition comprising an FGF21 compound and a GLP-1 compound, and a pharmaceutically acceptable carrier, wherein the GLP-1 compound:

i) comprises at least one of the following: DesaminoHis7, Aib8, Aib22, Arg26, Aib35, and/or Lys37;

ii) is a GLP-1 derivative comprising an albumin binding moiety which comprises at least one, preferably at least two, more preferably two, free carboxylic acid groups; or a pharmaceutically acceptable salt thereof;

iii) is a GLP-1 derivative comprising an albumin binding moiety that comprises an acyl radical of a dicarboxylic acid, preferably comprising a total of from 12 to 24 carbon atoms, such as C12, C14, C16, C18, C20, C22, or C24, most preferably C16, C18, or C20; wherein preferably a) the acyl radical is attached to the epsilon amino group of a lysine residue of the GLP-1 peptide via a linker; b) the linker comprises at least one OEG radical, and/or at least one Trx radical, and, optionally, additionally at least one Glu; and/or

iv) is selected from the compounds of claim 28, with the exception of compound G1; and/or the FGF21 compound comprising
(a) at least one of the following modifications as compared to SEQ ID NO:1: −1G, −1C, −1A, −1S, Q27E, Q28R, A31E, K56R, K59R, K69R, S71C, D102E, D102N, D102T, N121Q, des121N, N121D, K122R, D159E, L166F, S167G, M168L, V169aT, G170T, P171L, S172E, Q173A, G174A, G174V, Y179F, A180E, S181K and/or S181R; independently optionally with an N-terminal M (e.g., −1M); and/or
(b) an N-terminal extension as compared to SEQ ID NO:1 of up to 25 amino acid residues, preferably up to 20 amino acid residues, more preferably up to 15 amino acid residues, even more preferably up to 10 amino acid residues, or most preferably up to 6 amino acid residues, wherein at least 50%, preferably at least 60%, more preferably at least 70%, even more preferably at least 80%, or most preferably at least 90% of the N-terminally extending amino acid residues are G or S, with the proviso that said FGF21 analogue contains not more than 210 amino acid residues, preferably not more than 209 amino acid residues, more preferred not more than 206 amino acid residues and the further proviso that if the N-terminal extension consists of only a single amino acid, said amino acid is not Met, and a specific example of such a FGF21 compound is [−1A, 71C, 121Q, 166F, 167G, 168L, 171L, 172E, 173A, 174V, 179F, 180E, des181]FGF21.

### DEFINITIONS

The term “FGF21 compound” as used herein refers to native human FGF21 as well as analogues, fusion peptides, and derivatives thereof, which maintain FGF21 activity.

The sequence of the native human FGF21 protein is available from the UNIPROT database with accession no. Q9NSA1. The 209 amino acid precursor protein includes a signal peptide (amino acids 1-28) and a mature protein (amino acids 29-209). The mature protein is included herein as SEQ ID NO:1 (amino acids 1-181), and the signal peptide as SEQ ID NO:2 (amino acids 1-28). An isoform or allelic form of native human FGF21 having a Pro instead of Leu in the mature protein at position 146 of SEQ ID NO:1 herein is known from, i.a., US 2001012628 A1 (residue no. 174 of SEQ ID NO:2 in the published US application). Particular examples of native human FGF21 are the mature parts, viz. SEQ ID NO:1 and the L146P isoform thereof.

FGF21 activity may be determined using any method known in the art, e.g. the assay of Example 8 herein (glucose uptake in 3T3-L1 adipocytes).

The term “GLP-1 compound” as used herein refers to human GLP-1(7-37) (amino acids 1-31 of SEQ ID NO:3), exendin-4(7-45) (amino acids 1-39 of SEQ ID NO:4), as well as analogues, fusion peptides, and derivatives thereof, which maintain GLP-1 activity.

As regards position numbering in GLP-1 compounds: For the present purposes any amino acid substitution, deletion, and/or addition is indicated relative to the sequences of SEQ ID NO:3, and/or 4. However, the numbering of the amino acid residues in the sequence listing always starts with no. 1, whereas for the present purpose we want, following the established practice in the art, to start with amino acid residue no. 7 and assign number 7 to it. Therefore, generally, any reference herein to a position number of the GLP-1(7-37) or exendin-4 sequence is to the sequence starting with His at position 7 in both cases, and ending with Gly at position 37, or Ser at position 45, respectively.

GLP-1 activity may be determined using any method known in the art, e.g. the assay of Example 7 herein (stimulation of cAMP formation in a cell line expressing the human GLP-1 receptor).

The term “analogue” as used herein in the context of FGF21 as well as GLP-1 refers to polypeptides that are, or can be, deduced or derived from the respective FGF21, GLP-1, and exendin-4 sequence of SEQ ID NOs: 1, 3, and 4, respectively, by modification of the amino acid sequence thereof. Such modification may include substitution, deletion, and/or addition of one or more amino acids. For example, amino acids may be added and/or deleted at the C-terminus, the N-terminus, or internally in the amino acid sequence. Preferably amino acids are added and/or deleted at the C- and/or N-terminus, more preferably at the N-terminus. Amino acid sequences with C- or N-terminally deleted amino acids may also be referred to as truncated sequences, as is known in the art. Likewise, amino acids added internally in the sequence may be referred to as insertions. The term “variant” or “mutein” is now and then used herein instead of the term “analogue”.

Examples of FGF21 and GLP-1 analogues are disclosed in the particular embodiments section herein, in the experimental part, as well as in the claims.

The term “amino acid” or “amino acid residue” as referred to herein in the context of FGF21 and GLP-1 modifications includes the twenty standard alpha-amino acids being used by cells in protein biosynthesis and specified by the genetic code, viz. alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. The term also includes non-standard amino acids, such as selenocysteine and pyrrolysine which are also encoded by the genetic code but rare in proteins. Other non-standard amino acids found in proteins may be formed by post-translational modification, for example γ-carboxyglutamate and hydroxyproline. Additional examples of non-standard or non-natural amino acids which are not encoded by the genetic code are ornithine, and phosphoserine. Still further examples of non-standard amino acids are synthetic amino acids including amino acids manufactured by chemical synthesis, e.g. D-isomers of the amino acids encoded by the genetic code such as, D-alanine, D-glutamine, D-histidine, and D-leucine, Aib (α-aminoisobutyric acid), Abu (α-aminobutyric acid), Tle (tert-butylglycine), β-alanine, 3-aminomethyl benzoic acid, anthranilic acid, des-amino-histidine (abbreviated DesaminoHis (or DesaH), alternative name imidazopropionic acid, abbreviated Impr), the beta analogues of amino acids such as 3-alanine, 2-amino-histidine, 3-hydroxy-histidine, homohistidine, Nα-acetyl-histidine, α-fluoromethyl-histidine, α-methyl-histidine, α,α-dimethyl-glutamic acid, m-CF₃-phenylalanine (abbreviated m-CF₃-Phe), α,β-diaminopropionic acid (abbreviated Dap), 3-pyridylalanine, 2-pyridylalanine or 4-pyridylalanine, (1-aminocyclopropyl) carboxylic acid, (1-aminocyclobutyl) carboxylic acid, (1-aminocyclopentyl) carboxylic acid, (1-aminocyclohexyl) carboxylic acid, (1-aminocycloheptyl) carboxylic acid, and (1-aminocyclooctyl) carboxylic acid.

For the present purposes the two recognized codes of the standard amino acids (one-letter, and three-letter) are used interchangeably, or now and then the amino acid name is fully spelled out. These terms are of course considered fully equivalent (e.g. C=Cys=cysteine).

The term “derivative” as used herein in the context of FGF21 as well as GLP-1 refers to polypeptides which have been covalently modified. The term is not limiting as such, rather descriptive, as it is intended to mark a distinction between changes made to the constituent polypeptide compounds as such (“analogues”), and the covalent binding of a side chain to the polypeptide, whereby it becomes “derivatised”. If desired, the term derivative can be substituted with other general chemical terms, for example compound. Examples of derivatives include acylated and pegylated polypeptides, as is known in the art. Further examples of derivatives are disclosed in the particular embodiments section herein, in the experimental section, and in the claims.

The term “albumin binder” is also not intended to be limiting as such. Again, it is rather descriptive, as it reflects the overall aim or purpose of the side chain, viz. that the resulting compound (derivative) is capable of binding to human serum albumin which provides or at least contributes to a protracted effect often aimed at for the derivatives of the invention. If desired, this term can also be substituted with other general chemical terms, for example compound. Examples of albumin binders are disclosed in the particular embodiments section, the experimental part, and the claims.

Nomenclature: Analogues and derivatives are named herein using, interchangeably, polypeptide nomenclature, organic chemical nomenclature, and chemical formulas, or mixtures thereof, whatever is deemed best suited for easing the understanding of the technical matter in question.

Variant nomenclature: Variants of FGF21, GLP-1, and exendin-4 are named herein using, interchangeably, polypeptide nomenclature, organic chemical nomenclature, chemical formulas, amino acid sequences, or a mix thereof, whatever is deemed best suited for easing the understanding of the technical matter in question.

For example, a substitution in a variant may be indicated as: “Original amino acid-position-substituted amino acid”. The three or one letter code may be used. Accordingly, taking FGF21 as an example, the notation “K122C” or “Lys122Cys” means, that the FGF21 variant in question comprises a substitution of lysine with cysteine in the variant amino acid position corresponding to the amino acid at position 122 in FGF21 (SEQ ID NO:1). A substitution may, however, also simply be indicated as the position and the resulting amino acid residue, e.g. 122C is considered equivalent to K122C, as it refers to the same resulting molecule. If needed or desired, the position may be confirmed by aligning the variant and FGF21 as described further below (“alignment”).

Multiple substitutions may be separated by commas, and if desired surrounded by brackets in order to make it clear that they belong to the same variant. Taking again FGF21 as an example, the FGF21 analogue used for preparing compound F3 (Example 2) may for example be designated “K56R, K59R, K69R, K122R Met-FGF21”, or it may be referred to as “SEQ ID NO:1 with K56R, K59R, K69R, and K122R and an N-terminal M”. Alternatively, a “+” may be used to separate, as in the variant (−1M+56R+59R+69R+122R) of SEQ ID NO:1. Another example is GLP-1-variant (8V+22E) of SEQ ID NO:3, in which the Ala at position 8 (position 2 in the sequence listing) is substituted with Val, and Gly at position 22 (position 16 in the sequence listing) is substituted with Glu.

An extension can be described by reference to the actual SEQ ID NO by addition of position numbers (continued positive numbers in the C-terminal end, and negative numbers in the N-terminal end), or, more simply, by adding the amino acids of the extension in question, using the correct sequence thereof, to the compound in question, as for example in Met-FGF21 (−1M-FGF21).

For purposes of the present invention, the alignment of two related amino acid sequences, such as those of FGF21 and an analogue thereof, may be made using the Needle program from the EMBOSS package (http://emboss.org). A preferred version is 2.8.0. The Needle program implements the global alignment algorithm described in Needleman, S. B. and Wunsch, C. D. (1970) J. Mol. Biol. 48, 443-453. The substitution matrix used is BLOSUM62, gap opening penalty is 10, and gap extension penalty is 0.5.

In the alternative, the program “align” which is a Needleman-Wunsch alignment (i.e. a global alignment) may be used. The sequences are aligned by the program, using the default scoring matrix BLOSUM50. The penalty for the first residue of a gap is 12, and for further residues of a gap the penalties are 2. The Needleman-Wunsch algorithm is described in Needleman, S. B. and Wunsch, C D., (1970), Journal of Molecular Biology, 48: 443-453, and the align program by Myers and W. Miller in Optimal Alignments in Linear Space” CABIOS (computer applications in the biosciences) (1988) 4:11-17. “Align” is part of the FASTA package version v20u6 (see W. R. Pearson and D. J. Lipman (1988), “Improved Tools for Biological Sequence Analysis”, PNAS 85:2444-2448, and W. R. Pearson (1990) “Rapid and Sensitive Sequence Comparison with FASTP and FASTA,” Methods in Enzymology 183:63-98).

A pharmaceutical composition comprising an FGF21 compound and a GLP-1 compound of the invention may further comprise a pharmaceutically acceptable carrier. For injection, the carrier may be water, if desired supplemented with other materials, e.g., saline, such as physiological saline. Other pharmaceutically acceptable agents such as diluents and appropriate buffers may also be used. If desired, additional pharmaceutically acceptable agents such as emulsifiers, suspending agents, solvents, fillers, bulking agents, adjuvants, preservatives, antioxidants, colouring agents, and/or flavouring agents may also be used. The FGF21 and GLP-1 compounds may be used in the form of purified polypeptides, or formulated using appropriate pharmaceutically acceptable excipients, as is known in the art. The pharmaceutical composition may be administered in any way as is known in the art, e.g. injected, for example intravenously (i.v.), or subcutaneously (s.c.).

The FGF21 and GLP-1 compounds may be included in the pharmaceutical composition in therapeutically or prophylactically effective amounts. The amount depends upon the therapeutic or prophylactic objective, such as the condition of the patient in need of treatment, the desired route of administration, etc. The skilled medical practitioner may have to adjust dosage and modify the administration depending on these factors, as is routine in the art. Exemplary and non-limiting dosages are disclosed in the Examples.

Many of the FGF21 and GLP-1 compounds used according to this invention are known compounds. Those FGF21 and GLP-1 compounds used according to this invention which are not known compounds can be prepared analogously to the preparation of similar compounds.

### Particular Embodiments

The following are particular embodiments of the invention:

1. Use of an FGF21 compound and a GLP-1 compound in combination for the preparation of a medicament for the treatment of type 2 diabetes.

2. The use of embodiment 1, wherein the GLP-1 compound comprises the amino acid sequence of SEQ ID NO:3, SEQ ID NO:4, or is an analogue of SEQ ID NO:3 or 4 having a maximum of 15 amino acid substitutions, deletions, and/or additions.

3. The use of embodiment 2, wherein the GLP-1 compound comprises the amino acid sequence of SEQ ID NO:3, or is an analogue thereof having a maximum of 15 amino acid substitutions, deletions, and/or additions.

4. The use of embodiment 2, wherein the GLP-1 compound comprises the amino acid sequence of SEQ ID NO:4, or is an analogue thereof having a maximum of 15 amino acid substitutions, deletions, and/or additions; such as exenatide.

5. The use of any one of embodiments 1-4, wherein the GLP-1 compound has GLP-1 activity.

6. The use of embodiment 5, wherein the GLP-1 compound is as active, more active, or up to 10 times less active as compared to N-epsilon26-((S)-4-carboxy-4-hexadecanoylaminobutyryl)-[Arg34]GLP-1-(7-37) (compound G1), preferably up to 8, 6, 5, 4, 3 or 2 times less active, wherein the activity is preferably measured as the ability to stimulate formation of cAMP in a medium containing the human GLP-1 receptor, e.g. as described in Example 7.

7. The use of any one of embodiments 5-6, wherein an EC₅₀ value of the GLP-1 compound is determined based on an assay measuring the ability to stimulate formation of cAMP in a medium containing the human GLP-1 receptor, said EC₅₀ value preferably not exceeding 1000 pM, more preferably not exceeding 800, 600, 500, 400, 300, or 200 pM, wherein the determination preferably is performed as described in Example 7.

8. The use of any one of embodiments 2-7, wherein the maximum number of amino acid substitutions, deletions, and/or additions is 14, preferably 13, more preferably 12, even more preferably 11, or most preferably 10.

9. The use of any one of embodiments 2-8, wherein the maximum number of amino acid substitutions, deletions, and/or additions is 9, preferably 8, more preferably 7, even more preferably 6, or most preferably 5.

10. The use of any one of embodiments 2-9, wherein the maximum number of amino acid substitutions, deletions, and/or additions is 4, preferably 3, more preferably 2, or most preferably 1.

11. The use of any one of embodiments 2-3 and 5-10, wherein the GLP-1 compound comprises at least one of the following: DesaminoHis7, Aib8, Aib22, Arg26, Aib35, and/or Lys37.

12. The use of any one of embodiments 2-3 and 5-10, wherein the GLP-1 compound comprises at least one of the following: Glu22, and/or Arg34.

13. The use of any one of embodiments 2-3 and 5-12, wherein the GLP-1 compound comprises the following in combination: (34R), (8Aib+22Aib+35Aib+37K), (8Aib+34R), or (7DesaH+22E+26R+34R+37K).

14. The use of embodiment 2 and 4-10, wherein the GLP-1 compound comprises at least one of the following: 8V,G; 22E; 331; 36G; 37P; 38S; 39S; 40G; 41A; 42P; 43P; 44P; 45S; 46C; 46C-amide; preferably comprises the following in combination: (8V+22E), (8G+22E+36G), or (8V+22E+33I+36G+37P+38S+39S+40G+41A+42P+43P+44P+45S).

15. The use of any one of embodiments 1-14, wherein the GLP-1 compound is a fusion peptide with a second polypeptide, optionally via a linker.

16. The use of embodiment 15, wherein the second polypeptide is selected from the Fc portion of an immunoglobulin, human albumin, and analogues and fragments thereof.

17. The use of any one of embodiments 1-14, wherein the GLP-1 compound is a GLP-1 derivative.

18. The use of embodiment 17, wherein the GLP-1 derivative is modified at the C-terminal amino acid residue, or at an internal amino acid residue.

19. The use of embodiment 18, wherein the GLP-1 derivative is modified via the epsilon amino group of a lysine residue, such as 26K, or 37K.

20. The use of any one of embodiments 17-19, wherein the GLP-1 derivative comprises an albumin binding moiety.

21. The use of embodiment 20, wherein the albumin binding moiety comprises at least one, preferably at least two, more preferably two, free carboxylic acid groups, or a pharmaceutically acceptable salt thereof.

22. The use of any one of embodiments 17-21, wherein the albumin binding moiety comprises an acyl radical, such as acyl of fatty acids or dicarboxylic acids, for example hexadecanoyl- and 15-carboxypentadecanoyl-, the acyl radical preferably comprising a total of from 12 to 24 carbon atoms, such as C12, C14, C16, C18, C20, C22, or C24, most preferably C16, C18, or C20; or a pharmaceutically acceptable salt thereof.

23. The use of embodiment 22 wherein the acyl radical is attached to the epsilon amino group of a lysine residue of the GLP-1 peptide via a linker.

24. The use of embodiment 23, wherein the linker comprises at least one OEG radical (OEG is 8-amino-3,6-dioxaoctanic acid), at least one Trx radical (Trx is tranexamic acid, or trans-4-(amino-methyl)cyclohexanecarboxylic acid):

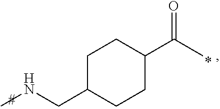

and/or at least one Glu (glutamine) radical.

25. The use of embodiment 24, wherein the linker comprises a di-OEG radical in which two OEG radicals have been combined via an amide bond.

26. The use of embodiment 25, wherein the linker further comprises a Glu radical, wherein preferably the amino group of Glu forms an amide bond with the acyl radical, and, more preferably, the gamma-acyl group of Glu forms an amide bond with the amino group of the di-OEG radical, the carboxyl group of which, most preferably, forms an amide bond with the epsilon-amino group of a Lys residue of the GLP-1 peptide.

27. The use of any one of embodiments 24-25, wherein the linker further comprises a Trx radical, wherein preferably the amino group of Trx forms an amide bond with the acyl radical, and, more preferably, the acyl group of Trx forms an amide bond with the amino group of Glu, the gamma-acyl group of which, even more preferably, forms an amide bond with the amino group of the di-OEG radical, the carboxyl group of which, most preferably, forms an amide bond with the epsilon-amino group of a Lys residue of the GLP-1 peptide. 28. The use of any one of embodiments 1-27, wherein the GLP-1 compound is selected from:

N-epsilon26-((S)-4-carboxy-4-hexadecanoylaminobutyryl)[Arg34]GLP-1-(7-37):

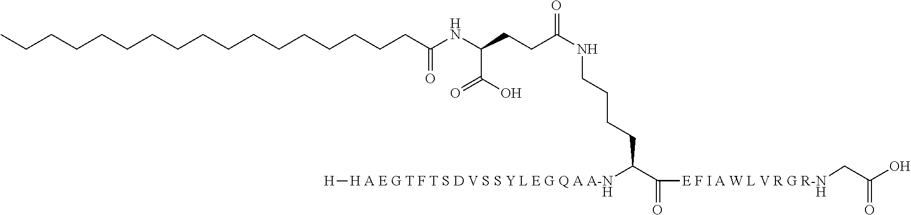

(compound G1);

N-epsilon37-[2-(2-{2-[2-(2-{2-[(S)-4-carboxy-4-({trans-4-[(19-carboxynonadecanoylamino)-methyl]cyclohexanecarbonyl}amino)butyrylamino]ethoxy}ethoxy)acetylamino]ethoxy}ethoxy)acetyl][De saminoHis7,Glu22,Arg26,Arg34,Lys37]GLP-1-(7-37):

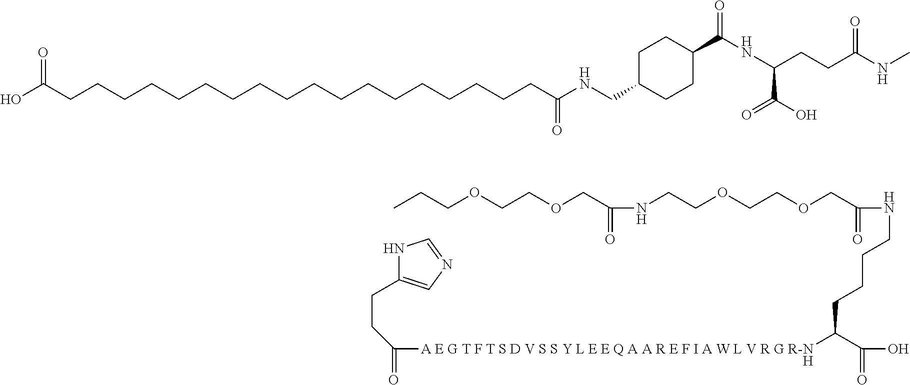

(compound G2);

N-epsilon26-[2-(2-{2-[2-(2-{2-[(S)-4-Carboxy-4-(17-carboxyheptadecanoylamino)butyryl-amino]ethoxy}ethoxy)acetylamino]ethoxy}ethoxy)acetyl][Aib8,Arg34]GLP-1-(7-37):

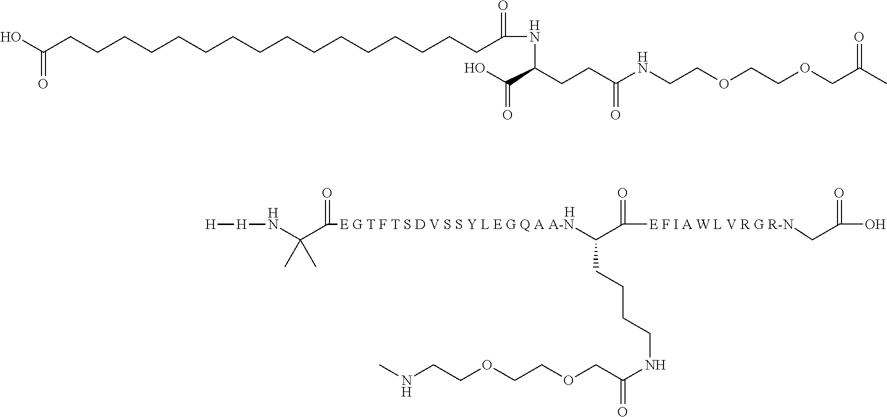

(compound G3);

N-epsilon37-[2-(2-{2-[2-(2-{2-[(S)-4-carboxy-4-(15-carboxypentadecanoylamino)butyryl-amino]ethoxy}ethoxy)acetylamino]ethoxy}ethoxy)acetyl] [Aib8,22,35,Lys37[GLP-1-(7-37):

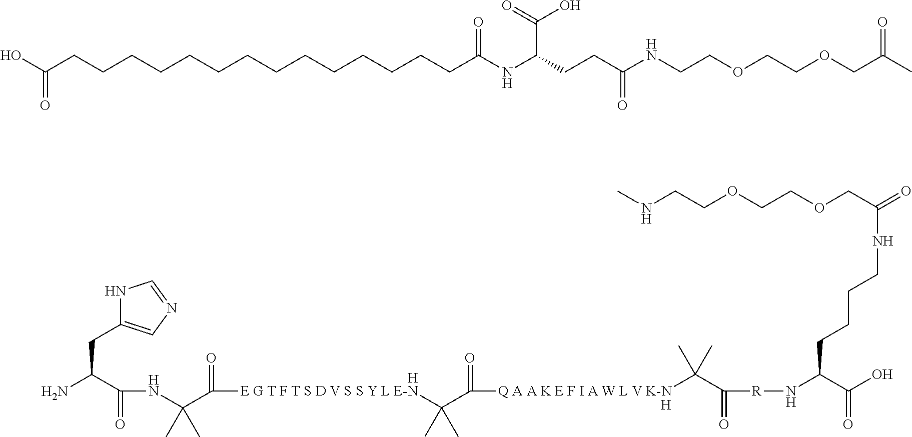

(compound G4);
and their pharmaceutically acceptable salts, amides, alkyls, or esters.

29. The use of any one of embodiments 1-28, wherein the FGF21 compound comprises the amino acid sequence of SEQ ID NO:1 or is an analogue thereof having a maximum of 30 amino acid substitutions, deletions, and/or additions.

30. The use of embodiment 29, wherein the FGF21 compound has FGF21 activity.

31. The use of embodiment 30, wherein the FGF21 compound has a potency of at least 1%, preferably at least 5%, more preferably at least 10%, even more preferably at least 20%, or most preferably at least 30% of the potency of Met-FGF21 (SEQ ID NO: 1 with an added N-terminal Met; compound F1), wherein the potency is determined by measuring glucose uptake in 3T3-L1 adipocytes.

32. The use of embodiment 31, wherein the potency is at least 40%, preferably at least 50%, more preferably at least 60%, even more preferably at least 70%, relative to the potency of Met-FGF21 (SEQ ID NO: 1 with an added N-terminal Met; compound F1).

33. The use of embodiment 32, wherein the potency is at least 80%, preferably at least 90%, more preferably at least 100%, even more preferably at least 110%, or most preferably at least 120%, relative to the potency of Met-FGF21 (SEQ ID NO: 1 with an added N-terminal Met; compound F1).

34. The use of any one of embodiments 31-33, wherein the potency is calculated as the EC₅₀ of the derivative relative to the EC₅₀ of Met-FGF21 (SEQ ID NO: 1 with an added N-terminal Met; compound F1).

35. The use of any one of embodiments 31-34, wherein the 3T3-L1 adipocytes derive from mouse 3T3-L1 fibroblasts, preferably ATCC CL-173, and the assay is preferably performed as described in Example 8.

36. The use of any of embodiments 29-35, wherein the maximum number of amino acid substitutions, deletions, and/or additions is 25, preferably 20, more preferably 15, even more preferably 10 and most preferably 9.

37. The use of any one of embodiments 29-36, wherein the maximum number of amino acid substitutions, deletions, and/or additions is 8, preferably 7, more preferably 6, even more preferably 5, and most preferably 4.

38. The use of any one of embodiments 29-37, wherein the maximum number of amino acid substitutions, deletions, and/or additions is 3, more preferably 2, or most preferably 1.

39. The use of any one of embodiments 29-38, wherein the FGF21 compound comprises at least one of the following: −1M, S71C, K56R, K59R, K69R, and/or K122R.

40. The use of any one of embodiments 29-38, wherein the FGF21 compound comprises at least one of the following: (118C+134C), 167A, (21C+33C), (26C+122C), 121A.

41. The use of any one of embodiments 1-40, wherein the FGF21 compound is an FGF21 derivative.

42. The use of embodiment 41, wherein the FGF21 derivative is modified at the N-terminal amino acid residue, or at an internal amino acid residue.

43. The use of embodiment 42, wherein the FGF21 derivative is modified via the amino group of the N-terminal amino acid residue, or via the thiol group of a cysteine residue, preferably an internal cysteine residue, such as 71C.

44. The use of any one of embodiments 41-43, wherein the FGF21 derivative comprises an albumin binding moiety.

45. The use of embodiment 44, wherein the albumin binding moiety comprises at least one, preferably at least two, more preferably two, free carboxylic acid groups, or a pharmaceutically acceptable salt thereof.

46. The use of any one of embodiments 41-45, wherein the albumin binding moiety comprises an acyl radical, such as acyl of fatty acids or dicarboxylic acids, for example 17-carboxy-heptadecanoyl- and 19-carboxynonadecanoyl-, the acyl radical preferably comprising a total of from 12 to 24 carbon atoms, such as C12, C14, C16, C18, C20, C22, or C24, most preferably C18, or C20; or a pharmaceutically acceptable salt thereof.

47. The use of embodiment 46 wherein the acyl radical is attached to the amino group of the N-terminal amino acid residue of the FGF21 peptide, e.g., to the amino group of -1M, or to the thiol group of an internal cysteine residue of the FGF21 peptide, e.g., to the thiol group of 71C, via a linker.

48. The use of embodiment 47, wherein the linker comprises at least one OEG radical (OEG is 8-amino-3,6-dioxaoctanic acid), and/or at least one Glu (glutamine) radical.

49. The use of embodiment 48, wherein the linker comprises a di-OEG radical in which two OEG radicals have been combined via an amide bond.

50. The use of embodiment 49, wherein the linker further comprises a Glu radical, wherein preferably the amino group of Glu forms an amide bond with the acyl radical, and, more preferably, the gamma-acyl group of Glu forms an amide bond with the amino group of the di-OEG radical, the carboxyl group of which, most preferably, forms an amide bond with the N-terminal amino group of the FGF21 peptide, or, alternatively, connects to the thiol group of a cysteine residue of the FGF21 peptide, optionally via a spacer, such as —N—(CH₂)₂—N—C(═O)—CH₂—.

51. The use of any one of embodiments 1-50, wherein the FGF21 compound is selected from:

the polypeptide having SEQ ID NO:1 (human FGF21);

the polypeptide having SEQ ID NO: 1 with an added N-terminal Met (Met-FGF21_human, compound F1);

S-71-({2-[2-(2-{2-[2-(2-{2-[(S)-4-carboxy-4-(19-carboxynonadecanoylamino)butyrylamino]-ethoxy}ethoxy)acetylamino]ethoxy}ethoxy)acetylamino]ethylcarbamoyl}methyl) [Cys71]Met-FGF21 (compound F2); and

N-alpha1-[2-(2-{2-[2-(2-{2-[(S)-4-carboxy-4-(17-carboxyheptadecanoylamino)butyrylamino]-ethoxy}ethoxy)acetylamino]ethoxy}ethoxy)acetyl] [Arg56, Arg59, Arg69, Arg122]-Met-FGF21 (compound F3);

and their pharmaceutically acceptable salts, amides, alkyls, or esters.

52. The use of any one of embodiments 1-51, wherein the GLP-1 compound is selected from the compounds of embodiment 28, and the FGF21 compound is selected from the compounds of embodiment 51.

53. The use of embodiment 52, wherein the FGF21 and GLP-1 compounds are:

the polypeptide having SEQ ID NO:1 (human FGF21), preferably with an added N-terminal Met (compound F1) together with N-epsilon26-[2-(2-{2-[2-(2-{2-[(S)-4-carboxy-4-(17-carboxyheptadecanoylamino)butyrylamino]ethoxy}ethoxy)acetylamino}ethoxy}ethoxy)acetyl][Aib8,Arg34]GLP-1-(7-37) (compound G3);

the polypeptide having SEQ ID NO:1 (human FGF21), preferably with an added N-terminal Met (compound F1) together with N-epsilon26-((S)-4-carboxy-4-hexadecanoylaminobutyryl)[Arg34]-GLP-1-(7-37) (compound G1); or

the polypeptide having SEQ ID NO:1 (human FGF21), preferably with an added N-terminal Met (compound F1) together with N-epsilon37-[2-(2-{2-[2-(2-{2-[(S)-4-carboxy-4-(15-carboxypentadecanoylamino)butyrylamino]ethoxy}ethoxy)acetylamino]ethoxy}ethoxy)acetyl] [Aib8,22,35,Lys37]GLP-1-(7-37) (compound G4);

or their pharmaceutically acceptable salts, amides, alkyls, or esters.

54. The use of any one of embodiments 1-53, wherein the compounds are administered simultaneously, and/or sequentially.

55. The use of any one of embodiments 1-54, wherein the viability of beta cells (INS-1) in the presence of 0.35 mM free fatty acids for cells pre-treated with the GLP-1 and FGF21 compounds in combination is at least 1.2 times (1.2×), preferably 1.4, more preferably 1.6, even more preferably 1.8, and most preferably at least 2.0 times the viability of cells pre-treated with each of the compounds alone under the same conditions, measured as absorbance in a MTT assay.

56. The use of embodiment 55 wherein i) the free fatty acids are prepared as described in Example 4; ii) the FGF21 and/or GLP-1 compounds are added to the cells one hour prior to exposure to the free fatty acids; iii) the cells are incubated for 48 hours before the viability is determined; iv) cell viability is measured as absorbance at 550 nm; and/or v) the conditions are generally as outlined in Example 4, e.g., by use of the Promega CellTiter96 kit.

57. The use of any one of embodiments 1-56, wherein the caspase activity of beta cells (INS-1) in the presence of 0.35 mM free fatty acids for cells pre-treated with the GLP-1 and FGF21 compounds in combination is below 90%, preferably below 80%, more preferably below 70%, even more preferably below 65% of the caspase activity of cells pre-treated with each of the compounds alone under the same conditions, measured as fluorescense in a Caspase-3/7 assay.

58. The use of embodiment 57, wherein i) the free fatty acids are prepared as described in Example 4; ii) the FGF21 and/or GLP-1 compounds are added to the cells one hour prior to exposure to the free fatty acids; iii) the cells are incubated for 48 hours before the caspase activity is determined; and/or iv) the conditions are generally as outlined in Example 4, e.g. by use of the Promega Apo-One homogenous Caspase-3/7 assay.

59. The use of any one of embodiments 1-58, wherein, in an acute study with db/db mice, the blood glucose (area under the curve, AUC) after sequential dosing of FGF21 and GLP-1 compounds is below 60%, preferably below 50%, even more preferably below 40%, or most preferably below 30% of the blood glucose (AUC) after dosing of each of the compounds alone under the same conditions.

60. The use of embodiment 59, wherein the AUC for the sequential dosing of FGF21 and GLP-1 compounds is below 90%, preferably below 80%, more preferably below 70%, and most preferably below 60% of the expected additive effect of the compounds.

61. The use of any one of embodiments 59-60, wherein i) the mice are dosed once daily with the FGF21 compound for 3 days; ii) the GLP-1 compound is administered one hour after the last dose of the FGF21 compound; iii) blood samples are taken and analysed for blood glucose (mmol/l) from 0-48 hours post dose of the GLP-1 compound; iv) the results are given as area under the glucose curve (AUC) based on all measurements (0-48 hours); and/or v) the conditions are generally as outlined in Example 5.

62. The use of any one of embodiments 60-61, wherein the expected additive effect is calculated using the following formula: AUC_{vehicle}−((AUC_{vehicle}−AUC_{FGF21})+(AUC_{vehicle}−AUC_{GLP-1})).

63. The use of any one of embodiments 1-62, wherein, in a subchronic study with db/db mice, the blood glucose (mmol/l) after simultaneous dosing of FGF21 and GLP-1 compounds is below 70%, preferably below 60%, even more preferably below 50%, or most preferably below 40% of the blood glucose after dosing of each of the compounds alone under the same conditions.

64. The use of embodiment 63, wherein i) the compounds are dosed twice daily for 21 days; ii) blood samples are taken and analysed for blood glucose (mmol/l) at day 0, 7, 14 and 21; and/or v) the conditions are generally as outlined in Example 6.

65. A combination of an FGF21 compound and a GLP-1 compound for the treatment of type 2 diabetes.

66. The combination of embodiment 65, to which each of the conditions of embodiments 2-64 are individually applied mutatis mutandis.

67. A composition comprising an FGF21 compound and a GLP-1 compound, and a pharmaceutically acceptable carrier, wherein the GLP-1 compound:

i) comprises at least one of the following: DesaminoHis7, Aib8, Aib22, Arg26, Aib35, and/or Lys37;

ii) is a GLP-1 derivative comprising an albumin binding moiety which comprises at least one, preferably at least two, more preferably two, free carboxylic acid groups; or a pharmaceutically acceptable salt thereof;

iii) is a GLP-1 derivative comprising an albumin binding moiety that comprises an acyl radical of a dicarboxylic acid, preferably comprising a total of from 12 to 24 carbon atoms, such as C12, C14, C16, C18, C20, C22, or C24, most preferably C16, C18, or C20; wherein preferably a) the acyl radical is attached to the epsilon amino group of a lysine residue of the GLP-1 peptide via a linker; b) the linker comprises at least one OEG radical, and/or at least one Trx radical, and, optionally, additionally at least one Glu; and/or

iv) is selected from the compounds of embodiment 28, with the exception of compound G1; and/or

the FGF21 compound:

a) comprises at least one of −1M, S71C, K56R, K59R, K69R, and/or K122R;

b) is an FGF21 derivative modified via the thiol group of a cysteine residue, preferably an internal cysteine residue, such as 71C;

c) is an FGF21 derivative comprising an albumin binding moiety;

d) is not a PEGylated FGF21 derivative;

e) is an FGF21 derivative comprising an albumin binding moiety which comprises at least one, preferably at least two, more preferably two, free carboxylic acid groups, or a pharmaceutically acceptable salt thereof;

f) is an FGF21 derivative comprising an albumin binding moiety that comprises an acyl radical, such as acyl of fatty acids or dicarboxylic acids, the acyl radical preferably comprising a total of from 12 to 24 carbon atoms, such as C12, C14, C16, C18, C20, C22, or C24, most preferably C18, or C20; or a pharmaceutically acceptable salt thereof; wherein preferably i) the acyl radical is attached to the amino group of the N-terminal amino acid residue of the FGF21 peptide, e.g. to the amino group of −1M, or to the thiol group of an internal cysteine residue of the FGF21 peptide, e.g. to the thiol group of 71C, via a linker; and preferably b) the linker comprises at least one OEG radical, and/or at least one Glu radical; and/or

g) is selected from the compounds of embodiment 51, with the exception of the polypeptide having SEQ ID NO: 1.

68. A composition comprising an FGF21 compound and a GLP-1 compound, and a pharmaceutically acceptable carrier, wherein the GLP-1 compound:

i) comprises at least one of the following: DesaminoHis7, Aib8, Aib22, Arg26, Aib35, and/or Lys37;

ii) is a GLP-1 derivative comprising an albumin binding moiety which comprises at least one, preferably at least two, more preferably two, free carboxylic acid groups; or a pharmaceutically acceptable salt thereof;

iii) is a GLP-1 derivative comprising an albumin binding moiety that comprises an acyl radical of a dicarboxylic acid, preferably comprising a total of from 12 to 24 carbon atoms, such as C12, C14, C16, C18, C20, C22, or C24, most preferably C16, C18, or C20; wherein preferably a) the acyl radical is attached to the epsilon amino group of a lysine residue of the GLP-1 peptide via a linker; b) the linker comprises at least one OEG radical, and/or at least one Trx radical, and, optionally, additionally at least one Glu; and/or

iv) is selected from the compounds of embodiment 28, with the exception of compound G1; and/or the FGF21 compound comprises:

(a) at least one of the following modifications as compared to SEQ ID NO:1: −1G, −1C, −1A, −1S, Q27E, Q28R, A31E, K56R, K59R, K69R, S71C, D102E, D102N, D102T, N121Q, des121N, N121D, K122R, D159E, L166F, S167G, M168L, V169aT, G170T, P171L, S172E, Q173A, G174A, G174V, Y179F, A180E, S181K and/or S181R; independently optionally with an N-terminal M (e.g., −1M); and/or

(b) an N-terminal extension as compared to SEQ ID NO:1 of up to 25 amino acid residues, preferably up to 20 amino acid residues, more preferably up to 15 amino acid residues, even more preferably up to 10 amino acid residues, or most preferably up to 6 amino acid residues, wherein at least 50%, preferably at least 60%, more preferably at least 70%, even more preferably at least 80%, or most preferably at least 90% of the N-terminally extending amino acid residues are G or S, with the proviso that said FGF21 analogue contains not more than 210 amino acid residues, preferably not more than 209 amino acid residues, more preferred not more than 206 amino acid residues and the further proviso that if the N-terminal extension consists of only a single amino acid, said amino acid is not Met.

69. The composition of embodiment 67 and 68, to which each of the conditions of embodiments 2-66 are individually applied mutatis mutandis.

70. The composition of any one of embodiments 67-69 which is a pharmaceutical formulation for the treatment of type 2 diabetes.

71. The composition of any one of embodiments 67-70, wherein the GLP-1 and FGF21 compounds are present in effective amounts.

72. The composition of any one of embodiments 67-71, to the extent possible, wherein the GLP-1 compound is as defined in said embodiments and the FGF21 compound is a derivative of [−1A, 71C, 121Q, 166F, 167G, 168L, 171L, 172E, 173A, 174V, 179F, 180E, des181]FGF21, for example, S-71-({2-[2-(2-{2-[2-(2-{2-[(S)-4-carboxy-4-(17-carboxyheptadecanoylamino)butyrylamino]ethoxy}-ethoxy)acetylamino]ethoxy}ethoxy)acetylamino]ethylcarbamoyl}methyl) [71C, 121Q, 166F, 167G, 168L, 171L, 172E, 173A, 174V, 179F, 180E, des181] Ala-FGF21.

73. A method of treating type 2 diabetes comprising administering to a patient an effective amount of an FGF21 compound and a GLP-1 compound in combination.

74. A method of improving the viability of beta cells comprising administering an effective amount of an FGF21 compound and a GLP-1 compound in combination.

75. A method of reducing apoptosis of beta cells comprising administering an effective amount of an FGF21 compound and a GLP-1 compound in combination.

76. A method of lowering blood glucose comprising administering an effective amount of an FGF21 compound and a GLP-1 compound in combination.

77. The method of any one of embodiments 73-76, to which each of the conditions of embodiments 2-66 are individually applied mutatis mutandis.

78. Compound N-epsilon37-[2-(2-{2-[2-(2-{2-[(S)-4-carboxy-4-(15-carboxypentadecanoyl-amino)butyrylamino]ethoxy}ethoxy)acetylamino]ethoxy}ethoxy)acetyl] [Aib8,22,35,Lys37]GLP-1-(7-37):

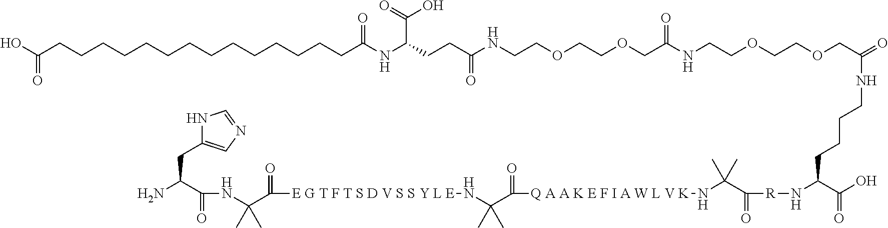

or a pharmaceutically acceptable salt, amide, alkyl, or ester thereof.

79. The compound of embodiment 77 which has GLP-1 activity, preferably as described in each of embodiments 6-7.

80. A composition comprising the compound of any one of embodiments 78-79 and a pharmaceutically acceptable carrier, preferably a pharmaceutical composition for treatment of type 2 diabetes, and wherein more preferably the compound is present in an effective amount.

81. The compound of any one of embodiments 78-79 for use as a medicament.

82. The compound of any one of embodiments 78-79 for use in the treatment of type 2 diabetes.

83. A method of treating type 2 diabetes comprising administering to a patient the compound of any one of embodiments 78-79.

84. A method of lowering blood glucose comprising administering the compound of embodiment 78.

85. Any novel feature or combination of features described herein.

Various references are cited herein, the disclosures of which are incorporated by reference in their entireties.

### EXAMPLES

The following examples serve to illustrate the invention.

### Abbreviations

Herein, the following abbreviations are used: DCM is dichloromethane, DIC is diisopropylcarbodiimide, DIPEA is diisopropylethylamine, DPBS is Dulbecco's Phosphate-Buffered Saline, DVB is divinyl benzene, EDAC is (3-dimethylaminopropyl)ethyl carbodiimide hydrochloride, fmoc is 9 H-fluoren-9-yl-methoxycarbonyl, h is hour(s), HEPES is 4-(2-hydroxyethyl)-1-piperazineethanesulfonic acid, HOAt is 1-hydroxy-7-azabenzotriazole, HOBt is 1-hydroxybenzotriazole, HPLC is High Performance Liquid Chromatography, IBMX is 3-isobutyl-1-methylxanthine, Inp is isonipecotic acid, IPTG is isopropyl β-D-1-thiogalactopyranoside check, LCMS is Liquid Chromatography Mass Spectroscopy, MALDI-TOF MS is Matrix-Assisted Laser Desorption/Ionization Time of Flight Mass Spectroscopy, MeOH is methanol, min is minutes, NanoES-MS is Nano-ElectroSpray tandem Mass Spectrometry, NMP is 1-methyl-pyrrolidin-2-one, OEG is 8-amino-3,6-dioxaoctanic acid, OtBu is tert. butyl ester, PBS is phosphate buffered saline, RT is room temperature, TFA is trifluoroacetic acid, THF is tetrahydrofuran, TIPS is triisopropylsilane, Tris is tris(hydroxymethyl)aminomethane or 2-amino-2-hydroxymethylpropane-1,3-diol, Trx is tranexamic acid, TSTU is O—(N-succimidyl)-N,N,N′,N′-tetramethyluronium tetrafluoroborate and UPLC is Ultra Performance Liquid Chromatography.

### General Methods

### LCMS Method 1 (LCMS1)

An Agilent Technologies LC/MSD TOF (G1969A) mass spectrometer was used to identify the mass of the sample after elution from an Agilent 1200 series HPLC system. The deconvolution of the protein spectra was calculated with Agilent's protein confirmation software.

### Eluents:

A: 0.1% Trifluoro acetic acid in water

B: 0.1% Trifluoro acetic acid in acetonitrile

Column: Zorbax 5u, 300SB-C3, 4.8×50 mm

Gradient: 25% -95% acetonitrile over 15 min

### LCMS Method 2 (LCMS2)

A Perkin Elmer Sciex API 3000 mass spectrometer was used to identify the mass of the sample after elution from a Perkin Elmer Series 200 HPLC system.

### Eluents:

A: 0.05% Trifluoro acetic acid in water

B: 0.05% Trifluoro acetic acid in acetonitrile

Column: Waters Xterra MS C-18×3 mm id 5 μm

Gradient: 5% -90% acetonitrile over 7.5 min at 1.5 ml/min

### LCMS Method 3 (LCMS3)

A Waters Micromass ZQ mass spectrometer was used to identify the mass of the sample after elution from a Waters Alliance HT HPLC system.

### Eluents:

A: 0.1% Trifluoro acetic acid in water

B: 0.1% Trifluoro acetic acid in acetonitrile

Column: Phenomenex, Jupiter C4 50×4.60 mm id 5 μm

Gradient: 10% -90% B over 7.5 min at 1.0 ml/min

### Example 1

### Preparation of GLP-1 Derivatives

The following GLP-1 compounds were prepared (all being derivatives of analogues of GLP-1(7-37) (SEQ ID NO:3)):

### Compound G1:

N-epsilon26-((S)-4-Carboxy-4-hexadecanoylaminobutyryl)[Arg34]GLP-1-(7-37), which may also be designated Arg³⁴Lys²⁶(Nε-(γ-glutamyl(Nα-hexadecanoyl)))-GLP-1(7-37)-OH:

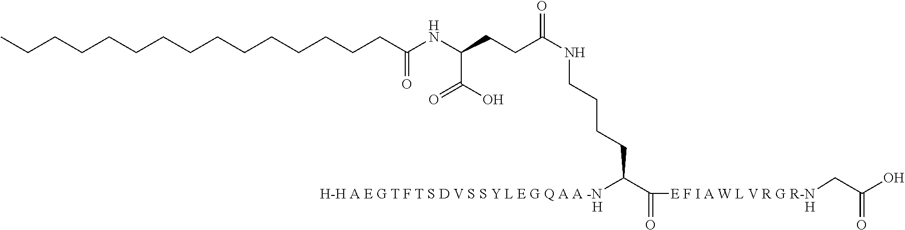

### Compound G2:

N-epsilon37-[2-(2-{2-[2-(2-{2-[(S)-4-Carboxy-4-({trans-4-[(19-carboxynonadecanoylamino)methyl]-cyclohexanecarbonyl}amino)butyrylamino]ethoxy}ethoxy)acetylamino]ethoxy}ethoxy)acetyl][Des-aminoHis7,Glu22,Arg26,Arg34,Lys37]GLP-1-(7-37):

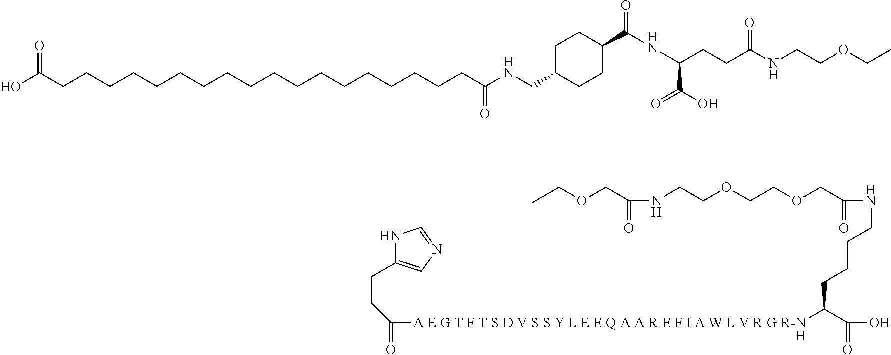

### Compound G3:

N-epsilon26-[2-(2-{2-[2-(2-{2-[(S)-4-Carboxy-4-(17-carboxyheptadecanoylamino)butyrylamino]ethoxy}-ethoxy)acetylamino]ethoxy}ethoxy)acetyl][Aib8,Arg34]GLP-1-(7-37)

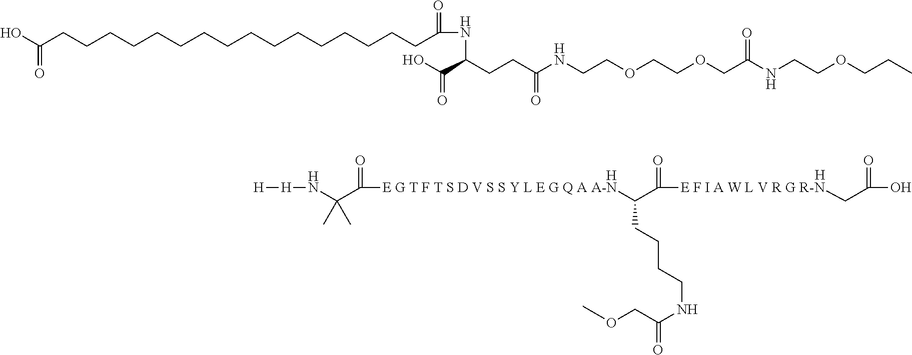

### Compound G4:

N-epsilon37-[2-(2-{2-[2-(2-{2-[(S)-4-Carboxy-4-(15-carboxypentadecanoylamino)butyrylamino]ethoxy}-ethoxy)acetylamino]ethoxy}ethoxy)acetyl] [Aib8,22,35,Lys37]GLP-1-(7-37)

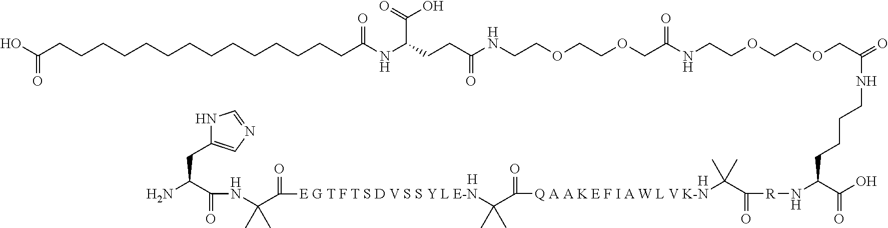

Compound G1 was prepared as described in Example 37 of WO 98/08871. Compound G2 was prepared as described in Example 26 of WO 09030771. Compound G3 was prepared as described in Example 4 of WO 2006/097537.

Novel compound G4 was prepared in similar fashion to the methods described in WO 09/030771, using a CEM Liberty peptide synthesizer.

LCMS: m/z=1046 (M/4)

Calculated (M)=4184.8

LCMS was performed on a setup consisting of Waters Acquity UPLC system and LCT Premier XE mass spectrometer from Micromass. The HPLC pump was connected to two eluent reservoirs containing:

A: 0.1% Formic acid in water

B: 0.1% Formic acid in acetonitrile

The analysis was performed at room temperature (RT) by injecting an appropriate volume of the sample (preferably 2-10□I) onto the column which was eluted with a gradient of A and B.

The HPLC conditions, detector settings and mass spectrometer settings used are given in the following table:

Column: Waters Acquity UPLC BEH, C-18, 1.7 □m, 2.1 mm×50 mm

Gradient: 5%-95% acetonitrile linear during 4.0 min at 0.4 ml/min

Detection: 214 nm (analogue output from DAD (Diode Array Detector))

MS ionisation mode: API-ES (atmospheric pressure ionisation electrospray), Scan 100-2000 amu (atomic mass units), step 0.1 amu

### Example 2

### Preparation of FGF21 Compounds

The following FGF21 compounds were prepared:

### Compound F1:

Native human FGF21 (SEQ ID NO:1), however with an N-terminal Met due to expression in E. coli, i.e., Met-FGF21_human.

### Compound F2:

The S71C analogue of compound F1 was modified at position 71 with the following reagent:

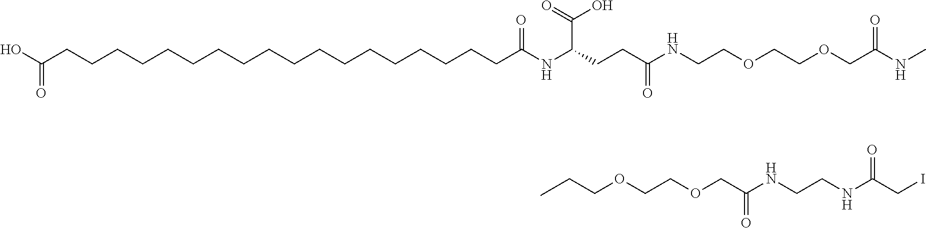

resulting in the compound S-71-({2-[2-(2-{2-[2-(2-{2-[(S)-4-carboxy-4-(19-carboxynonadecanoyl-amino)butyrylamino]ethoxy}ethoxy)acetylamino]ethoxy}ethoxy)acetylamino]ethylcarbamoyl}methyl)-[Cys71]Met-FGF21.

### Compound F3:

The K56R, K59R, K69R, K122R analogue of compound F1 was modified at the N-terminal Met residue with the following reagent:

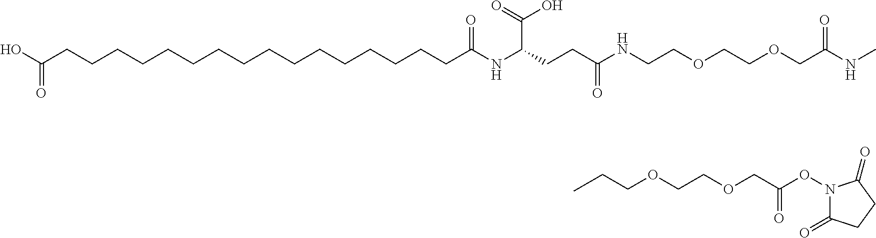

resulting in the compound N-alpha1-[2-(2-{2-[2-(2-{2-[(S)-4-carboxy-4-(17-carboxyheptadecanoylamino)butyrylamino]ethoxy}ethoxy)acetylamino]ethoxy}ethoxy)acetyl][Arg56, Arg59, Arg69, Arg122]-Met-FGF21.

Compounds F1, F2, and F3 were prepared as described in PCT/EP2010/—(see in particular Examples 1, 3, 4, 6, and 7), which claims priority from EP09151227.7 of 23 Jan. 2009 and EP 09152144.3 of 5 Feb. 2009. The relevant Example text of the latter EP priority application is inserted below.

The DNA and amino acid sequences for human FGF21 have been disclosed by, e.g., Nishimura et al. in Biochim. Biophys. Acta 1492(1):203-206 (2000). The sequences are also available from public databases with accession nos. EMBL:AB021975 and UNIPROT:Q9NSA1, respectively.

The native polypeptide is synthesised with a signal peptide of 28 amino acids for secretion:

| | |
|---|---|
| 1 | MDSDETGFEH SGLWVSVLAG LLLGACQAHP IPDSSPLLQF |
| | GGQVRQRYLY |
| 51 | TDDAQQTEAH LEIREDGTVG GAADQSPESL LQLKALKPGV |
| | IQILGVKTSR |
| 101 | FLCQRPDGAL YGSLHFDPEA CSFRELLLED GYNVYQSEAH |
| | GLPLHLPGNK |
| 151 | SPHRDPAPRG PARFLPLPGL PPALPEPPGI LAPQPPDVGS |
| | SDPLSMVGPS |
| 201 | QGRSPSYAS |

The signal peptide, shown in italics above, is included in the appended sequence listing as SEQ ID NO:2. The mature FGF21 polypeptide consisting of the remaining 181 amino acids is included in the sequence listing as SEQ ID NO:1.

The mature FGF21 polypeptide was cloned and expressed as an intracellular protein in E. coli, without the signal peptide, but with an added N-terminal methionine. More in particular, a 550 by coding region including at the 3′-end the ATG codon for Met, as well as Nde1 and BamH1 restriction sites at the 3′- and 5′-ends, respectively, was inserted into the expression vector pET 11c in Nde1-BamH1 under control of the phage T7 promoter, and transformed into E. coli B BL21(DE3). The cells were grown in LB amp 100 ug/mL to OD₄₅₀ 0.5, and expression was induced with 0.3 mM IPTG for 4 hours at 37° C. Crude extracts of cells were made by sonication for analysis of FGF21 expression.

A Coomassie stained SDS-PAGE gel showed successful expression of FGF21 which was identified mainly in the soluble supernatant fraction, with very little in the insoluble pellet. Although the calculated MW of the thus expressed FGF21 (Met-FGF21) (Compound A) is 19.5 kD, it migrated on the gel as a 25 kD protein, which is likely due to the high content of prolines, delaying the movement of the protein.

The FGF21 polypeptide and its analogues were further purified as follows:

A slurry (20% w/v) of E. coli in 10 mM potassium phosphate buffer pH 7.5 was sonicated (3 seconds on/off intervals on ice for 5 minutes). The polypeptide was pelleted by centrifugation (10,000×g, for 30 minutes), re-solubilised by sonication in 50 mM Tris pH 8.0, and debris removed by centrifugation (10,000×g, for 30 minutes). The polypeptide in the resulting supernatant was purified by anion exchange chromatography (50 mM Tris pH 8.0, 50-250 mM NaCl) using Q Sepharose Fast Flow resin (GE Healthcare), as generally described in Protein Purification. Principles and Practice Series: Springer Advanced Texts in Chemistry Scopes, Robert K. 3rd ed., 1994. In some instances, further purification was done by size exclusion chromatography using a HiLoad 26/60 Superdex pg 75 column (GE Healthcare) operated with 50 mM Tris pH 8.0 and 200 mM NaCl. For storage the polypeptide was transferred to 50 mM ammonium bicarbonate pH 7.9, lyophilized, and kept at −80° C.

Albumin binders containing a maleimide may be synthesised as described in the following, and FGF21 and analogues thereof containing a free cysteine may be derivatised with such albumin binders as also described in the following.

Preparation of 17-((S)-1-carboxy-3-{2-[2-({2-[2-({2-[3-(2,5-dioxo-2,5-dihydropyrrol-1-yl)-propionylamino]ethylcarbamoyl}methoxy)ethoxy]ethylcarbamoyl}methoxy)ethoxy]ethylcarbamoyl}-propylcarbamoyl)heptadecanoic acid:

### Step 1: fmoc-ethylenediamine 2-chlorotrityl resin

5.8 g (7.5 mmol) 2-Chlorotrityl chloride resin (100-200 mesh, 1% DVB, loaded 1.3 mmol/g) was swollen in DCM (80 mL) for ca 1 h and then it was drained. Fmoc-ethylene diamine hydrogen chloride was suspended in NMP (30 mL) and DCM (30 mL) and DIPEA (5 eq, 6.42 mL). This suspension was added to the resin and shaken for 3 h. The resin was drained and washed with 17:2:1, DCM:MeOH:DIPEA, DCM, NMP and DCM (3×80 mL). It was dried over KOH/NaOH in a dessicator.

### Step 2: fmoc-OEG-ethylenediamine 2-chlorotrityl resin

3 mmol of the fmoc-ethylenediamine 2-chlorotrityl resin was modified using a CEM Liberty microwave peptide synthesizer and fmoc-based solid-phase peptide methodology. The resin was swollen in NMP (60 mL) and drained.

The resin was FMOC deprotected using 5% piperidine in NMP (60 mL), heated for 30 sec, drained, washed with NMP (60 ml), followed by additional 5% piperidine in NMP (60 mL), heated for 3 min at 70-75° C., followed by washing with NMP (4×60 mL). A 0.3 M solution of Fmoc-8-amino-3,6-dioxaoctanic acid+0.3 M HOAT in NMP (45 mL) was added to the resin followed by addition of a 0.75 M solution of DIC in NMP (18 mL). The reaction was heated to 70-75° C. for 10 min, followed by a wash with NMP (4×60 mL).

### Step 3: fmoc-OEG-OEG-ethylenediamine 2-chlorotrityl resin

The resin was FMOC deprotected using 5% piperidine in NMP (60 mL), heated for 30 sec, drained, washed with NMP (60 ml), followed by additional 5% piperidine in NMP (60 mL), heated for 3 minutes at 70-75° C. followed by washing with NMP (4×60 mL). A 0.3 M solution of Fmoc-8-amino-3,6-dioxaoctanic acid+0.3 M HOAT in NMP (45 mL) was added to the resin, followed by addition of a 0.75 M solution of DIC in NMP (18 mL). The reaction was heated to 70-75° C. for 10 min followed by a wash with NMP (4×60 mL).

### Step 4: fmoc-gamma-Glu-OEG-OEG-ethylenediamine 2-chlorotrityl resin

The resin was FMOC deprotected using 5% piperidine in NMP (60 mL), heated for 30 sec, drained, washed with NMP (60 ml), followed by additional 5% piperidine in NMP (60 mL), heated for 3 min at 70-75° C., followed by washing with NMP (4×60 mL). A 0.3M solution of Fmoc-Glu-OtBu+0.3 M HOAT in NMP (45 mL) was added to the resin, followed by addition of a 0.75M solution of DIC in NMP (18 mL). The reaction was heated to 70-75° C. for 10 min, followed by a wash with NMP (4×60 mL).

### Step 5: C18-diacid-gamma-Glu-OEG-OEG-ethylenediamine 2-chlorotrityl resin

The resin was FMOC deprotected using 5% piperidine in NMP (60 mL), heated for 30 sec, drained, washed with NMP (60 ml), followed by additional 5% piperidine in NMP (60 mL), heated for 3 min at 70-75° C., followed by washing with NMP (4×60 mL). A 0.3M solution of octadecanedioic acid mono-tert-butyl ester+0.3 M HOAT in NMP (45 mL) was added to the resin, followed by addition of a 0.75M solution of DIC in NMP (18 mL). The reaction was heated to 70-75° C. for 10 min, followed by a wash with NMP (4×60 mL).

### Step 6: 17-[(S)-3-(2-{2-[(2-{2-[(2-aminoethylcarbamoyl)methoxy]ethoxy}ethylcarbamoyl)-methoxy]ethoxy}ethylcarbamoyl)-1-carboxypropylcarbamoyl]heptadecanoic acid

The resin was treated with TFA/TI PS/water 95:2.5:2.5 for 1 h. The resin was filtered off and the filtrate was concentrated under vacuum. Acetonitrile was added and the sample was re-concentrated. The crude product was purified by HPLC (10-50% acetonitrile, 0.1% TFA, 60 mL/min, C18, 50 mm×200 mm, 15A). LCMS2 m/z: 777 (M+1).

### Step 7: 17-((S)-1-carboxy-3-{2-[2-({2-[2-({2-[3-(2,5-dioxo-2,5-dihydropyrrol-1-yl)propionyl-amino]ethylcarbamoyl}methoxy)ethoxy]ethylcarbamoyl}methoxy)ethoxy]ethylcarbamoyl}propyl-carbamoyl)heptadecanoic acid

N-maleoyl-beta-alanine (0.65 mmol, 110 mg) was dissolved in NMP. EDAC (0.65 mmol, 125 mg) and HOBt (0.65 mmol, 88 mg) were added, and the mixture was stirred for 1 h at RT. A solution of 17-[(S)-3-(2-{2-[(2-{2-[(2-aminoethylcarbamoyl)methoxy]ethoxy}ethylcarbamoyl)methoxy]ethoxy}ethyl-carbamoyl)-1-carboxypropylcarbamoyl]heptadecanoic acid (0.65 mmol, 504 mg) in NMP (5 ml) was added, and the mixture was stirred for 16 h at RT. The crude product was purified by HPLC (25-65% acetonitrile, 0.1% TFA, 60 mL/min, C18, 50 mm×200 mm, 15A) to yield 200 mg of the title compound. LCMS2 m/z: 927.8 (M+1).

Preparation of the K122C Met-FGF21 derivative S-122-[1-(2-{2-[2-(2-{2-[2-(2-{2-[(S)-4-carboxy-4-(17-carboxyheptadecanoylamino)butyrylamino]ethoxy}ethoxy)acetylamino]ethoxy}ethoxy)-acetylamino]ethylcarbamoyl}ethyl)-2,5-dioxopyrrolidin-3-yl] [Cys122]-Met-FGF21:

The Cys residue at position 122 in the K122C Met-FGF21 analogue, prepared as described above (SEQ ID NO:1 with K122C and an N-terminal M), was modified at the thiol group with the following reagent, which was prepared as described above:

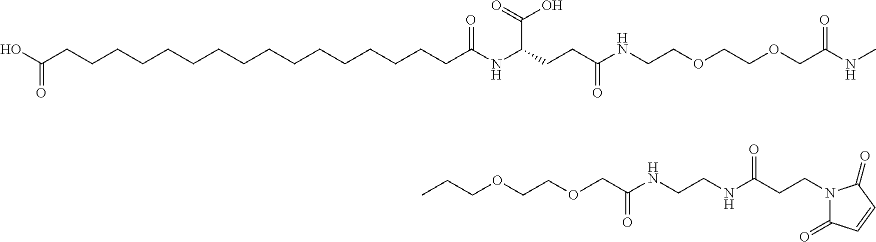

[Cys122]-Met-FGF21 (lyophilized) was dissolved in 20 mM Tris buffer pH 7.5 and buffer exchanged to 20 mM Tris buffer using PD-10 columns (GE Healthcare 170851-01). To 7 ml (1.48 μmol) of this solution (4.1 mg/ml) was added 1.5 ml of a solution containing 17-((S)-1-carboxy-3-{2-[2-({2-[2-({2-[3-(2,5-dioxo-2,5-dihydropyrrol-1-yl)propionylamino]ethylcarbamoyl}methoxy)ethoxy]ethyl-carbamoyl}methoxy)ethoxy]ethylcarbamoyl}propylcarbamoyl)heptadecanoic acid in acetonitrile/Tris buffer (1.3:1) (2.96 μmol). The reaction was allowed to react at RT for 1 h. The reaction mixture was filtered through a 0.22 um filter and was purified using a size exclusion chromatography (GE Healthcare, Superdex 200, 26/60) eluting with 20 mM Tris buffer pH 7.5, followed by ion exchange chromatography (Mono-Q 5/50, gradient from 0-0.5 M NaCl in 20 mM Tris, pH 7.5 over 60 column volumes). After analysis by LCMS and SDS-PAGE the relevant fractions were pooled and buffer exchanged to 50 mM NH₄HCO₃ and lyophilized. LCMS1: Theoretical mass=20442.2, found 20442.3.

The S71C Met-FGF21 derivative S-71-({2-[2-(2-{2-[2-(2-{2-[(S)-4-carboxy-4-(19-carboxynonadecanoylamino)butyrylamino]ethoxy}ethoxy)acetylamino]ethoxy}ethoxy)acetylamino]ethyl-carbamoyl}methyl)[Cys71]Met-FGF21 was prepared as follows:

Preparation of 19-{(S)-1-carboxy-3-[2-(2-{[2-(2-{[2-(2-iodoacetylamino)ethylcarbamoyl]-methoxy}ethoxy)ethylcarbamoyl]methoxy}ethoxy)ethylcarbamoyl]propylcarbamoyl}nonadecanoic acid:

### Step 1: 19-[(S)-3-(2-{2-[(2-{2-[(2-Aminoethylcarbamoyl)methoxy]ethoxy}ethylcarbamoyl)-methoxy]ethoxy}ethylcarbamoyl)-1-tert-butoxycarbonylpropylcarbamoyl]nonadecanoic acid tert-butyl ester

To a solution of 19-{(S)-1-tert-butoxycarbonyl-3-[2-(2-{[2-(2-carboxymethoxyethoxy)ethyl-carbamoyl]methoxy}ethoxy)ethylcarbamoyl]propylcarbamoyl}nonadecanoic acid tert-butyl ester (500 mg) in acetonitrile (15 ml) was added TSTU (224 mg) and DIPEA (0.13 ml). After stirring for 2 h at RT this mixture was pored into a solution of ethylenediamine (0.50 ml) in acetonitrile (5 ml). After stirring for 2 h the mixture was concentrated in vacuo. The residue was stirred in 1 N NaOH (100 ml) and ethyl acetate (400 ml). The layers were separated. The organic layer was dried with magnesium sulphate and concentrated in vacuo to give a white solid. This solid was stirred in ethanol and then filtrated. The filtrate was concentrated to give a syrup. Yield: 250 mg (48%). LCMS3: Theoretical mass: 916.26 Found: 926.7.

### Step 2: 19-{(S)-1-tert-Butoxycarbonyl-3-[2-(2-{[2-(2-{[2-(2-iodoacetylamino)ethylcarbamoyl]-methoxy}ethoxy)ethylcarbamoyl]methoxy}ethoxy)ethylcarbamoyl]propylcarbamoyl}nonadecanoic acid tert-butyl ester

To a solution of iodoacetic acid (60 mg) in DCM (8 ml) was added TSTU (90 mg) and DIPEA (0.05 ml). After stirring at RT for 60 min a solution of 19-[(S)-3-(2-{2-[(2-{2-[(2-aminoethylcarbamoyl)-methoxy]ethoxy}ethylcarbamoyl)methoxy]ethoxy}ethylcarbamoyl)-1-tert-butoxycarbonylpropylcarbamoyl]nonadecanoic acid tert-butyl ester (0.25 g) in DCM (8 ml) and DIPEA (0.05 ml) was added. After stirring for 120 min, the mixture was diluted with DCM (100 ml) and 1 N HCl (50 ml) was added. The layers were separated. The organic layer was dried with magnesium sulphate and concentrated in vacuo. The residue was co-concentrated with ethanol to give a solid compound. Yield 225 mg (76%). LCMS3: Theoretical mass: 1084.2 Found: 1084.8

### Step 3: 19-{(S)-1-Carboxy-3-[2-(2-{[2-(2-{[2-(2-iodoacetylamino)ethylcarbamoyl]methoxy}-ethoxy)ethylcarbamoyl]methoxy}ethoxy)ethylcarbamoyl]propylcarbamoyl}nonadecanoic acid

19-{(S)-1-tert-Butoxycarbonyl-3-[2-(2-{[2-(2-{[2-(2-iodoacetylamino)ethylcarbamoyl]methoxy}-ethoxy)ethylcarbamoyl]methoxy}ethoxy)ethylcarbamoyl]propylcarbamoyl}nonadecanoic acid tert-butyl ester (225 mg) was treated with TFA (10 ml) for 90 min. The mixture was concentrated in vacuo and co-concentrated with toluene twice. The residue was purified by HPLC using A-buffer: 0.1% TFA in water and B-buffer: 0.1% TFA in acetonitrile. Gradient 10-80% B over 45 min. Flow: 20 ml/min, C18 column 30 mm×250 mm, 110A. Yield 45 mg (22%). LCMS3: Theoretical mass: 971.98 Found: 972.6.

The Cys residue at position 71 in the S71C Met-FGF21 analogue, prepared as generally described above (SEQ ID NO:1 with S71C and an N-terminal M), was modified at the thiol group with the following reagent:

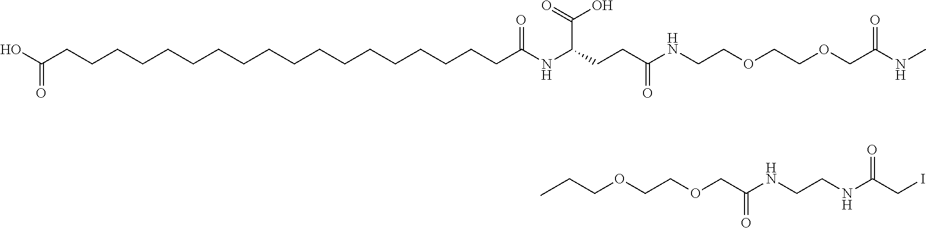

[Cys71] Met-FGF21 (7.53 mg, 385 nmol), freeze dried from NH₄HCO₃, was dissolved in 3×350 ul 0.02M TRIS, pH 7.8, and buffer exchanged through PD 10 columns to 0.02M TRIS, pH 7.8. Approximately 3.5 ml was collected. 19-{(S)-1-Carboxy-3-[2-(2-{[2-(2-{[2-(2-iodoacetylamino)ethyl-carbamoyl]methoxy}ethoxy)ethylcarbamoyl]methoxy}ethoxy)ethylcarbamoyl]propylcarbamoyl}-nonadecanoic acid (1.5 mg), which was prepared as described above, was dissolved in 0.02M TRIS, pH 7.8 buffer/acetonitrile 1:1 (0.75 ml). To the solution of [Cys71] Met-FGF21 was added iodo acetamide solution (0.561 ml, 3 eq). The acetonitrile concentration was 7%. The mixture was left at RT for 70 h. The mixture was ultra filtrated in Amicon Ultra-4 centrifugal device MWCO 10000 at 4000 g for 10 min. Ultrafiltration with approximately 4 ml A-buffer was repeated for another 4 times to remove reagent. The sample was purified by anion exchange on a monoQ 5/50 GL column using A-buffer: 20 mM TRIS, pH 7.8; B-buffer: 20 mM TRIS, 50 mM NaCl, pH 7.8, flow 0.5 ml and a gradient from 0-100% B over 60CV. The isolated fractions containing product were pooled and concentrated by ultracentrifugation in Amicon Ultra-4 centrifugal device MWCO 10000 at 6000 rpm for 2×10 min.

A buffer exchange to 50 mM NH₄HCO₃ was made using PD 10 (GE 179851-01) columns. Approximately 4.0 ml eluate was collected. This was filtered through a Millex GV sterile 0.22 um filter and freeze dried. Yield 2.18 mg. LCMS1: Theoretical mass: 20400.2 Found: 20400.13.

The K56R, K59R, K69R, K122R Met-FGF21 derivative N-alpha1-[2-(2-{2-[2-(2-{2-[(S)-4-carboxy-4-(17-carboxyheptadecanoylamino)butyrylamino]ethoxy}ethoxy)acetylamino]ethoxy}ethoxy)-acetyl][Arg56, Arg59, Arg69, Arg122]-Met-FGF21 was prepared as follows:

The N-terminal Met residue in the K56R, K59R, K69R, K122R Met-FGF21 analogue, prepared as generally described above (SEQ ID NO:1 with K56R, K59R, K69R, and K122R and an N-terminal M), was modified at the alpha amino group with the following reagent:

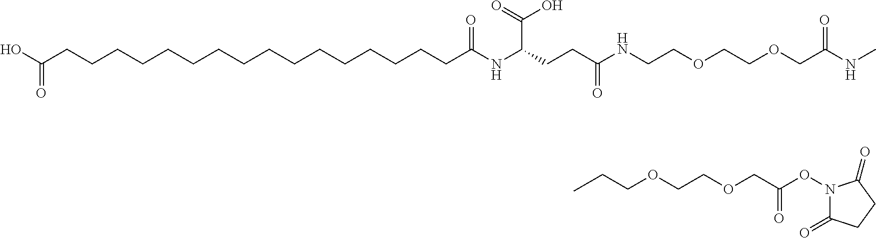

[Arg56, Arg59, Arg69, Arg122]-Met-FGF21 (lyophilized) was dissolved in DPBS buffer and buffer exchanged to DPBS buffer using PD-10 columns (GE Healthcare170851-01) yielding 3.5 ml (4.3 mg/ml, 0.77 μmol). The sample was diluted with DPBS buffer (10.5 ml) and a solution of 17-((S)-1-carboxy-3-{2-[2-({2-[2-(2,5-dioxopyrrolidin-1-yloxycarbonylmethoxy)ethoxy]ethylcarbamoyl}methoxy)-ethoxy]ethylcarbamoyl}propylcarbamoyl)heptadecanoic acid (6.2 μmol), which was prepared as generally described above, in acetonitrile (7.5 ml) was added. After 1 h at RT, the mixture was cooled to 0° C. and cold 0.2 M NaOH (21 ml) was added. After 30 min at 0° C. the mixture was neutralized with hydrochloric acid. The mixture was concentrated using Amicon Centriprep ultracel YM10 centrifugal filters (10000 MWCO), then diluted with 5 ml 20 mM Tris buffer, pH 7.5 and re-concentrated twice (final volume approximately 5 ml). The solution mixture was filtered through a 0.22 um filter and was purified by ion exchange chromatography and lyophilized as described above. LCMS1: Theoretical mass: 20368.1 Found: 20367.2.

### Example 3

### Restoration of Glucose Stimulated Insulin Release Ex Vivo

This ex vivo example investigates the ability of pancreatic islets from diabetic db/db mice to restore, in response to treatment with FGF21 and GLP-1 compounds, the ability to release insulin in response to glucose stimulation.

Islets from 25 db/db mice (Charles River), 15 weeks of age, were isolated according to the following procedure:

Animals were killed by cervical dislocation and fixated with pins on a Styrofoam plate. Pancreata were removed and transferred to a Packard vial containing 5 ml collagenase (Life Science, grade II, cat. no. 100502) 300 units/ml (one pancreas/vial) which was kept on ice until all pancreata were removed. Then the pancreata were shaken in the Grant/Edmund S25 thermoshaker at 200 strokes/min. at 37° C. for 5 min. The tissue was transferred to a fresh vial containing 5 ml 150 units/ml collagenase (supernatant was discarded) and shaken again in the thermoshaker for 5 min. The tissue was further shaken 3-5 min. on the thermoshaker, and thereafter all shaking is done by hand for 5-10 sec. each time. These steps were repeated until all tissue was digested. The process was followed continuously under the stereomicroscope and once digested the tissue was pooled and collected in progressively fewer tubes. All supernatants were washed 3 times in HBSS+0.5% NCS (HBSS (10×) from Gibco, cat. no. 14060, is diluted 10× in H₂O before use, Newborn Calf Serum (NCS) from Gibco, cat. no. 26010-066), allowed to sediment for 5 min. between each wash, and left on ice, following which the islets were ready for picking. Islets were purified by transferring a little bit of supernatant to a Petri dish, filling up with HBSS+NCS and subsequently transferring (by mouth pipetting) with a constriction pipette to a new Petri dish. From there the islet were re-picked until pure, and then incubated in RPMI 1640 medium (Gibco, cat no 61870-010)+10% NCS at 37° C.

The day after the isolation the islets from all animals were mixed and divided into three portions and incubated with 50 nM FGF21 compound (Compound F1), 100 nM GLP-1 compound (Compound G3), and buffer (control), respectively, for 48 hours. After incubation, the islets were washed in Krebs Ringer solution (115 mM NaCl, 4.7 mM KCl, 2.6 mM CaCl₂, 1.2 mM KH₂PO₄, 1.2 mM MgSO₄, 10 mM HEPES, 0.2% BSA, 2 mM glutamine, 5 mM NaHCO₃, pH 7.4) and subsequently used in an islet perifusion experiment (Suprafusion 2500 from Brandel). For each of the three preincubation conditions four perifusion columns each with 60 islets were used. The islets were placed in Bio-gel P2 (Bio-Rad. cat. no. 150-4114) in the columns with a nylon mesh below and above the gel. The islets were initially perifused for 40 minutes with Krebs Ringer solution containing 3 mM glucose to obtain a stable baseline of insulin release. After 40 minutes the buffer was exchanged to 15 mM glucose for 30 minutes (time 40-70 min). Then the buffer was exchanged to 15 mM glucose containing 10 μM forskolin (Tocris 1099, batch 3A/86008) for 40 minutes (time 70-110 min). Finally the buffer was exchanged to low glucose (3 mM) for 30 minutes (time 110-140 min). The buffer was set to flow through the perifusion system at a rate of 0.3 ml/minute. Samples were taken every 5 minutes from time 30 to 140 min. Samples were stored at −20° C. and subsequently analysed for insulin, essentially as described by Poulsen et al. in Journal of Biomolecular Screening 12(X); p. 1-8, 2007 (LOCI (Luminescent Oxygen Channeling Immunoassay) sandwich immunoassay).

The results (ng/ml insulin) are shown in Table 1 below as mean+/−standard error of mean (SEM). The figures for the control and the FGF21 compound are based on three repetitions (due to problems with one of the four columns in these two experiments), while the figures for the GLP-1 compound are based on four repetitions.

TABLE 1

| Time (min) | Control | FGF21 compound | GLP-1 compound |
|---|---|---|---|
| 35 | 0.62 +/− 0.02 | 0.92 +/− 0.10 | 0.67 +/− 0.07 |
| 40 | 0.68 +/− 0.08 | 0.71 +/− 0.06 | 0.60 +/− 0.00 |
| 45 | 0.60 +/− 0.00 | 0.68 +/− 0.04 | 0.61 +/− 0.01 |
| 50 | 6.24 +/− 1.81 | 19.56 +/− 6.74 | 10.46 +/− 0.77 |
| 55 | 13.60 +/− 0.87 | 28.33 +/− 2.09 | 33.43 +/− 2.08 |
| 60 | 3.84 +/− 0.13 | 12.07 +/− 0.50 | 16.15 +/− 1.41 |
| 65 | 3.06 +/− 0.17 | 11.13 +/− 0.66 | 12.51 +/− 1.43 |
| 70 | 3.55 +/− 0.28 | 13.37 +/− 0.63 | 11.92 +/− 1.10 |
| 75 | 4.48 +/− 0.19 | 13.47 +/− 0.78 | 11.98 +/− 1.64 |
| 80 | 50.67 +/− 3.82 | 77.17 +/− 14.84 | 52.48 +/− 3.93 |
| 85 | 121.33 +/− 4.81 | 160.00 +/− 16.52 | 107.20 +/− 9.16 |
| 90 | 124.33 +/− 9.94 | 154.00 +/− 15.00 | 105.78 +/− 11.06 |
| 95 | 104.90 +/− 8.56 | 148.33 +/− 17.95 | 91.55 +/− 8.28 |
| 100 | 89.57 +/− 6.74 | 125.00 +/− 17.35 | 77.10 +/− 6.46 |
| 105 | 76.47 +/− 6.54 | 99.40 +/− 11.84 | 63.10 +/− 5.04 |
| 110 | 62.43 +/− 6.39 | 79.97 +/− 8.43 | 51.85 +/− 4.04 |
| 115 | 47.30 +/− 4.13 | 69.63 +/− 9.61 | 40.88 +/− 2.49 |
| 120 | 37.63 +/− 2.80 | 56.23 +/− 8.37 | 32.23 +/− 2.39 |
| 125 | 19.97 +/− 2.48 | 30.03 +/− 6.67 | 15.58 +/− 1.79 |
| 130 | 7.42 +/− 1.12 | 13.16 +/− 3.21 | 5.32 +/− 1.01 |
| 135 | 3.32 +/− 0.57 | 5.76 +/− 0.70 | 2.87 +/− 0.61 |
| 140 | 2.53 +/− 0.46 | 3.92 +/− 0.37 | 1.86 +/− 0.27 |

The corresponding Area Under the Curve (designated AUC) figures were also calculated, and for the statistics the Student's T-test was used. The AUC results for the time intervals of time=35-75 minutes (response to glucose increase) and time=75-140 minutes (forskolin response) are shown in Tables 2 and 3, respectively.

TABLE 2
AUC for time 35-75 minutes (glucose response)

| Control | FGF21 | GLP-1 |
|---|---|---|
| 157.9; 155.5; 198.3 | 521.2; 514.5; 359.9 | 450.5; 528.5; 492.9; 367.8 |
| (mean 170.6) | (mean 465.2**) | (mean 459.9**) |

The FGF21 AUC result is significantly higher than the control (p=0.0057, corresponding to two asterisks (**)), and this is also the case for the GLP-1 AUC result as compared to the control (p=0.0010, also corresponding to two asterisks (**)).

TABLE 3
AUC for time 75-140 minutes (forskolin response)

| Control | FGF21 | GLP-1 |
|---|---|---|
| 4258; 3457; 3517 | 5402; 4177; 5832 | 3339; 4005; 3037; 2674 |
| (mean 3744) | (mean 5137*) | (mean 3239) |

This means that the FGF21 AUC result may be higher than the control (p=0.067 indicates a tendency, but without significance). This does not appear to be the case for the GLP-1 AUC result (p=0.28). However, the AUC result for FGF21 is significantly higher than that for GLP-1 (p=0.02) corresponding to the one asterisk (*) used in Table 3.

In conclusion, the FGF21 compound as well as the GLP-1 compound are capable of restoring glucose stimulated insulin release ex vivo from db/db mice pancreatic islets (Table 2 results). This is first of all an indication of a potential usefulness of these compounds for treatment of diabetes type 2 with a direct positive effect on the pancreatic islets.

Turning then to the forskolin results, forskolin is an adenylate cyclase activator and it serves to raise levels of cyclic AMP (cAMP). cAMP is an important signal carrier necessary for the proper biological response of cells to hormones and other extracellular signals. It is required for cell communication in the hypothalamus/pituitary gland axis and for the feedback control of hormones. It acts by activating protein kinase A.

Furthermore, it is well-known that one of the effects of incretins like GLP-1 actually is generation of cAMP.

The results of Table 3 (showing that FGF21 incubated islets respond significantly better than the GLP-1 incubated islets to forskolin), therefore may be taken as a pointer to a potential synergistic effect of FGF21 and GLP-1 in combination (FGF21 provides a bonus effect on the insulin release, as compared to the cAMP formation effect provided by GLP-1).

### Example 4

### Protection of Beta-Cells from Apoptosis Ex Vivo

This ex vivo example investigates the influence of FGF21 and GLP-1 compounds on FFA (free fatty acid) induced apoptosis of rat insulinoma beta-cells (INS-1).

Compound F1 (Prospec, cat. no. CYT-474) was used as the FGF21 compound, and compound G1 was used as the GLP-1 compound.

INS-1 cells were seeded in 96-well plates (50000 cells/well) and incubated overnight in cell medium (RPMI 1640 medium (Gibco, cat. no. 61870-010), supplemented with 10% FCS (Gibco, cat. no. 10085-140), 1% Pen/Strep (Gibco, cat. no. 15140-114) and 0.5 ml beta-mercaptoethanol (Gibco, cat. no. 31350-010 (50 mM)). The FGF21 compound (50 nM) and the GLP-1 compound (50 nM) were added to the cells 1 hour before the cells were to be exposed to FFA. The FFA's were prepared as follows: Stock solutions of palmitic acid (Sigma P5585) and oleic acid (Sigma 01383) of 1M in DMSO were mixed 1:2 and heated to 60 degree Celsius. The FFA mix was then diluted 10 times in 0.1M NaOH. This mixture was then further diluted in cell medium to yield a FFA concentration of 1 mM. The FFAs were then added to the cells to a final concentration of 0.35 mM. The cells were incubated for 48 hours and then the following three assays were performed: 1) Cell viability was assessed using the CellTiter 96® Non-radioactive Cell Proliferation Assay (MTT) from Promega, performed according to the manufacturer's instruction (absorbance at 550 nm, A550); 2) Apoptosis was assessed using the Apo-ONE® Homogeneous Caspase-3/7 Assay from Promega, performed according to the manufacturer's instruction (fluorescence); and 3) Insulin accumulated in the medium during the 48 hour incubation with FFA was analysed (as described in Example 3; ng/ml insulin).

The results of 1), 2) and 3) are shown in Tables 4, 5, and 6 below, respectively, all figures being mean+/−standard error of mean (SEM). All experiments were set up with four repetitions, but some ended up being made only in triplo due to experimental errors (viz. for viability, 0.35 mM FFA: GLP-1; for apoptosis, 0.35 mM FFA: control and FGF21, 0.00 mM FFA: GLP-1; and for accumulated insulin, 0.35 mM FFA: control, FGF21 and GLP-1, 0.00 mM FFA: control).

TABLE 4
Cell viability (MTT assay, A550)

| | | | | FGF21 and GLP-1 |
|---|---|---|---|---|
| | | GLP-1 | FGF21 | compounds in |
| FFA (mM) | Control | compound | compound | combination |
| 0.00 | 1.17 +/− 0.03 | 1.10 +/− 0.03 | 1.19 +/− 0.05 | 1.08 +/− 0.05 |
| 0.35 | 0.20 +/− 0.01 | 0.29 +/− 0.01 | 0.28 +/− 0.01 | 0.61 +/− 0.10 |

Table 4 shows that the cell viability in the absence of free fatty acids does not significantly differ between the four treatments (FFA=0.00 mM). On the other hand, the cell viability in the presence of free fatty acids (FFA=0.35 mM) is significantly improved by each of GLP-1 and FGF21 alone, as well as by the combination thereof, relative to the control (p=0.0014, 0.0031, and 0.0057, respectively, corresponding to a two asterisks significance level for each (**, Student's t-test)).

Furthermore, interestingly, the cell viability in the combination experiment is significantly improved as compared to each of GLP-1 and FGF21 alone (p=0.042, and 0.016, respectively, corresponding to a one asterisk significance level for each (*, student's t-test)). Accordingly, the combination of the FGF21 and GLP-1 compounds better than each of the compounds alone protects the beta cells (INS-1) from apoptosis induced by free fatty acids, measured as cell viability in a MTT assay.

TABLE 5
Apoptosis (Caspase 3/7 assay, fluorescense)

| | | | | Combination |
|---|---|---|---|---|
| | | | | of FGF21 |
| FFA | | GLP-1 | FGF21 | and GLP-1 |
| (mM) | Control | compound | compound | compounds |
| 0.00 | 2718 +/− 146 | 3389 +/− 209 | 1827 +/− 22 | 3017 +/− 622 |
| 0.35 | 43672 +/− 815 | 42056 +/− 2745 | 33278 +/− 1254 | 27307 +/− 2241 |

Caspases, or cysteine-aspartic acid proteases, are a family of cysteine proteases, which play an essential role in apoptosis (programmed cell death). A rise in caspase activity is therefore indicative of increased apoptosis.

The results in Table 5 show that in the absence of free fatty acids (FFA=0.00 mM) the caspase level is the same in each of the four treatment groups. The presence of free fatty acids on the other hand (FFA=0.35 mM) leads to an increase in caspase activity in all four groups. However, the caspase activity in the FGF21 group is significantly smaller than in the control group (p=0.002, corresponding to a two asterisks significance level (**, Student's t-test), and also as compared to the GLP-1 group (p=0.049, corresponding to a one asterisk significance level (*, Student's t-test)).

Furthermore, the caspase activity in the group receiving the combination treatment is significantly lower as compared to the GLP-1 group (p=0.0059, corresponding to a two asterisks significance level (**, Student's t-test)). Also as compared to the control, the combination differs significantly (two asterisks, **, p=0.002, Student's t-test). Finally, while these results do not allow a significant conclusion as regards the combination treatment versus FGF21 alone (p=0.09, Student's t-test), there may be a tendency of a lowering also here.

Accordingly, the combination of the GLP-1 and FGF21 compounds is better than the GLP-1 compound alone and the control reduces the caspase 3/7 activity and thereby apoptosis.

TABLE 6
Insulin (ng/ml)

| | | | | FGF21 and GLP-1 |
|---|---|---|---|---|
| | | GLP-1 | FGF21 | compounds in |
| FFA (mM) | Control | compound | compound | combination |
| 0.00 | 689 +/− 79 | 1463 +/− 147 | 1011 +/− 112 | 1888 +/− 132 |
| 0.35 | 385 +/− 33 | 1045 +/− 55 | 569 +/− 14 | 1207 +/− 110 |

Table 6 shows that, in the absence of free fatty acids (FFA=0.00 mM) the GLP-1 compound alone, and the combination lead to a significantly increased insulin secretion as compared to the control (p=0.009 corresponding to two asterisks (**), and p=0.0009 corresponding to three asterisks (***), respectively). The numerical increase observed for FGF21 alone is not significant (p=0.08, not significant (ns)).

Still in the absence of free fatty acids, the insulin level in the group receiving the combination is significantly higher, relative to FGF21 alone (p=0.002 corresponding to two asterisks (**)), but only numerically higher as compared to GLP-1 alone (p=0.08, ns).

In the presence of free fatty acids (FFA=0.35 mM), the GLP-1 compound, the FGF21 compound, and the combination thereof lead to a significantly increased insulin secretion as compared to the control (p=0.0005 corresponding to three asterisks (***), p=0.007 corresponding to two asterisks (**), and p=0.0016 corresponding to two asterisks (**), respectively).

Still in the presence of free fatty acids (FFA=0.35 mM), the insulin level in the group receiving the combination treatment is significantly higher as compared to FGF21 alone (p=0.0046 corresponding to two asterisks (**)), but only numerically higher as compared to GLP-1 alone (p=0.30, ns).

These results reflect insulin accumulated over 48 hours. Without wishing to be bound by any theory, the reason why accumulated insulin is higher in the presence of free fatty acids may be 1) that more cells survive due to the presence of the compounds (FGF21 and GLP-1) and are thus capable of releasing insulin; and/or 2) the compounds may stimulate the individual cell to release more insulin. For GLP-1, reason 1) as well as 2) contribute to the effect. For FGF21, at least reason 1) contributes to the effect, cf. the MTT and caspase results of Tables 4 and 5. The FGF21 result in the absence of free fatty acids confirms that this compound does not stimulate insulin release. That nevertheless such effect is seen in the presence of free fatty acids may be due to the ability of FGF21 to improve cell viability, viz. reason 1), again without wishing to be bound by this theory.

In conclusion, these results confirm that the cells which survive the presence of free fatty acids are functional, due to the presence of the FGF21 and the GLP-1 compounds.

### Example 5

### Effect on Blood Glucose in Vivo, Acute Study in db/db Mice

This acute in vivo study investigates the effect of FGF21 and GLP-1 compounds on blood glucose levels of db/db mice.

The db/db mouse is a hyperglycaemic, hyperinsulinaemic, hyperphagic and obese model of type 2 diabetes.

The following study design was used:

23 db/db mice (Male, C57BLKS db/db, from Taconic, Denmark, 15-16 weeks of age).

Dosed subcutaneously 1 × daily with 0.125 mg/kg human FGF21 (n=11) (compound F1) or vehicle (n=12) for 3 days. Vehicle was sterile PBS-buffer (DPBS, Gibco, cat. no. 14190).

Both groups of animals were then dosed with either vehicle or GLP-1 compound G4 at 1 nmol/kg (n=5-6). The G4 compound is a GLP-1 analogue which has an extended half-life due to its derivatisation with an albumin binder. The GLP-1 or vehicle dose was given subcutaneously one hour after the last FGF21 or vehicle dose.

Blood samples of 10 μl were taken from the tip of the tail for the measurement of blood glucose from 0-48 hours post dose of the GLP-1 compound.

Results were analysed using the area under the glucose curve (AUC) for the vehicle group, the GLP-1 group, the FGF21 group and the combined group.

Table 7 below displays the AUC_{0-48 h} for the 4 different groups.

TABLE 7
Glucose lowering capacity (AUC)

| | | Vehicle/ | FGF21 | FGF21/ | |
|---|---|---|---|---|---|
| | Vehicle/ | GLP-1 | compound/ | GLP-1 | |
| Group | Vehicle | compound | Vehicle | compounds | Expected |
| N | 6 | 6 | 6 | 5 | |
| Mean | 533.5 | 347.9 | 389.8 | 109.5** | 204.0 |
| (mM × min) | | | | | |
| Std. error | 45.6 | 56.6 | 72.9 | 24.6 | |

**p = 0.0022 (Students T-test, comparison to “Expected”)
Two-way ANOVA, testing for interaction: p = 0.40

The column to the right displays the expected AUC if there was an additive efficacy of the two compounds. The expected AUC was calculated using the following formula:

AUC_{vehicle}−((AUC_{vehicle}−AUC_{FGF212})+(AUC_{vehicle}−AUC_{GLP-1}))

As this expected AUC was significantly higher than the observed effect of the combination, it is concluded that there is a synergistic effect between the two compounds. However, testing for interaction using Two-way ANOVA yielded an insignificant result. This may be due to the relatively small sample size.

Accordingly, a once-daily treatment with the FGF21 compound for three days followed by one dose of the GLP-1 compound leads to an unexpected bonus effect as regards lowering of blood glucose.

### Example 6

### Effect on Blood Glucose in Vivo, Subchronic Study in db/db Mice

This subchronic in vivo dosing study investigates the effect of GLP-1 and FGF21 compounds on blood glucose. The study was performed in db/db mice (as described in Example 5).

The design of the study was:

48 db/db mice, male, 11-12 weeks of age at study start.

Dosed subcutaneously 2 × daily with either (i) 0.5 mg/kg human FGF21 (compound F1; n=12); (ii) 30 nmol/kg of GLP-1 compound G1 (n=12); (iii) a combination of 0.5 mg/kg of compound F1 and 30 nmol/kg of compound G1 (n=12); or (iv) vehicle (sterile PBS buffer, n=12).

All groups were dosed for 21 days.

Blood samples of 10 μl were taken from the tip of the tail for blood glucose measurements once weekly.

Table 8 below displays the blood glucose values (mmol/l) during the subchronic dosing study, as measured on day 0, 7, 14 and 21.

TABLE 8
Blood glucose (mmol/l)

| | | | | FGF21 and GLP-1 |
|---|---|---|---|---|
| | | FGF21 | GLP-1 | compounds |
| Group | Vehicle | compound | compound | in combination |
| Day 0 | 21.1 ± 0.9 | 19.9 ± 1.2 | 18.7 ± 0.9 | 21.1 ± 1.6 |
| Day 7 | 20.3 ± 1.0 | 17.2 ± 1.3 | 11.0 ± 1.0 | 6.3 ± 0.7 |
| Day 14 | 23.7 ± 1.6 | 16.2 ± 1.8 | 12.3 ± 0.8 | 5.5 ± 0.3 |
| Day 21 | 26.0 ± 1.4 | 19.2 ± 3.0 | 16.7 ± 1.7 | 5.7 ± 0.4 |

Two-way ANOVA, testing for interaction between the effect of the FGF21 compound and the GLP-1 compound at day 7, 14 and 21 showed no significant interaction.

One-way ANOVA, testing for the effect of the FGF21 compound, the GLP-1 compound and the combination, respectively, versus vehicle, showed significant effects of both compounds at all time points, except on day 7 for the FGF21 compound alone.

One way ANOVA comparing the FGF21 compound alone vs the combination thereof with the GLP-1 compound showed a significantly better effect of the combination on day 7, 14 and 21 (p=0.0001, all days). Comparing the GLP-1 compound alone vs. the combination thereof with the

FGF21 compound also showed a significantly better effect of the combination at day 7 (p=0.01), day 14 (p=0.001) and day 21 (p=0.0001).

Accordingly, treatment with the FGF21 and GLP-1 compounds in combination provides a statistically significant improvement in blood glucose as compared to treatment with each of the compounds alone.

### Example 7

### GLP-1 Activity Assay—Stimulation of cAMP Formation in a Cell Line Expressing the Cloned Human GLP-1 Receptor

The following assay may be used to determine the activity (potency) of GLP-1 compounds. In brief, the ability of GLP-1 compounds to stimulate formation of cyclic AMP (cAMP) in a medium containing the human GLP-1 receptor is measured.

In principle, purified plasma membranes from a stable transfected cell line, BHK467-12A (tk-ts13), expressing the human GLP-1 receptor are stimulated with the GLP-1 compound in question, and the potency of cAMP production is measured using the AlphaScreen™ cAMP Assay Kit from Perkin Elmer Life Sciences.

The cells are grown at 5% CO₂ in DMEM, 5% FCS, 1% Pen/Strep (Penicillin/Streptomycin) and 0.5 mg/ml of the selection marker G418.

Cells at approximate 80% confluence are washed 2× with PBS (Phosphate Buffered Saline) and harvested with Versene (aqueous solution of the tetrasodium salt of ethylenediaminetetraacetic acid), centrifuged 5 min at 1000 rpm and the supernatant removed. The additional steps are all made on ice. The cell pellet is homogenized by the Ultrathurax mixed for 20-30 sec. in 10 ml of Buffer 1 (20 mM Na-HEPES, 10 mM EDTA, pH=7.4), centrifuged 15 min at 20.000 rpm and resuspended in 10 ml of Buffer 2 (20 mM Na-HEPES, 0.1 mM EDTA, pH=7.4). The suspension is homogenized for 20-30 sec and centrifuged 15 min at 20.000 rpm. Suspension in Buffer 2, homogenization and centrifugation is repeated once and the membranes are resuspended in Buffer 2 and ready for further analysis or stored at −80° C.

The functional receptor assay is carried out by measuring the peptide induced cAMP production by The AlphaScreen Technology. The basic principle of The AlphaScreen Technology is a competition between endogenous cAMP and exogenously added biotin-cAMP. The capture of cAMP is achieved by using a specific antibody conjugated to acceptor beads. Formed cAMP is counted and measured at an AlphaFusion Microplate Analyzer. The EC₅₀ values are calculated, e.g. using the Graph-Pad Prism software (version 5).

The EC₅₀ values may be indicated relative to, e.g., the EC₅₀ for compound G1. The EC₅₀ values of compounds G2 and G3 relative to that of compound G1 were about 5 times, and 3 times higher, respectively, while the EC50 value of the compound of SEQ ID NO: 4 was about 0.3 times that of compound G1.

### Example 8

### FGF21 Activity Assay—Glucose Uptake in 3T3-L1 Adipocytes

The following assay may be used for determining the biological activity, or potency, of FGF21 compounds.

Mouse 3T3-L1 fibroblasts (e.g. available from ATCC, catalogue no. CL-173) are maintained in basal medium (DMEM (4500 mg/I Glucose) with 10% Fetal Bovine Serum (FBS) and Penicillin/Streptomycin). The cells are not allowed to reach confluence and should be passed (transferred to new vials) before reaching approx. 60% of confluency (by visual inspection).

For the glucose uptake assay, cells are plated 80,000 cells/well in a 24 well plate, or 20,000 cells/well in a 96 well plate, and when they reach confluency (high density, with a view to have differentiated adipose cells made), the medium is changed from basal medium to basal medium containing Troglitazone, IBMX, Dexamethasone (commercially available from, e.g., Sigma) and human insulin (commercially available from, e.g., Novo Nordisk A/S).

The cells are used 7-14, preferably 7-10, days after initiation of differentiation. The cells are stimulated with increasing concentrations (0-300 nM) of the FGF21 compounds for 20 hours in basal medium. Before addition of 3H-deoxyglucose (in what follows: the tracer) the cells are washed in warm (approximately 37° C.) assay buffer (PBS with 1 mM MgCl₂ and 2 mM CaCl₂), HEPES and 0.1% Human serum albumin) and the cells are incubated with the tracer for 1 hour. This incubation is terminated by washing twice in ice cold assay buffer. The cells are lysed with Triton X-100 and lysates transferred to a 96 wells plate, microscint-40 (commercially available from, e.g., Perkin Elmer) is added and amount of tracer counted in a TOP-counter (e.g. a Packard top-counter from Perkin Elmer).

The EC₅₀ of the FGF21 compound in question is calculated, and may be indicated relative to that of, e.g., compound F1. The EC₅₀ of compound F2 and F3 relative to that of compound F1 were 11%, and 30%, respectively.

1. A method of treating type 2 diabetes comprising administering an FGF21 compound and a GLP-1 compound in combination.

2. The method of claim 1, wherein the GLP-1 compound comprises the amino acid sequence of SEQ ID NO:3, SEQ ID NO:4, or is an analogue of SEQ ID NO:3 or 4 having a maximum of 15 amino acid substitutions, deletions, and/or additions.

3. The method of claim 1, wherein the GLP-1 compound is a GLP-1 derivative which comprises an albumin binding moiety.

4. The method of claim 3, wherein the albumin binding moiety comprises at least one, preferably at least two, more preferably two, free carboxylic acid groups, or a pharmaceutically acceptable salt thereof.

5. The method of claim 3, wherein the albumin binding moiety comprises an acyl radical, such as acyl of fatty acids or dicarboxylic acids, for example hexadecanoyl- and 15-carboxy-pentadecanoyl-, the acyl radical preferably comprising a total of from 12 to 24 carbon atoms, such as C12, C14, C16, C18, C20, C22, or C24, most preferably C16, C18, or C20; or a pharmaceutically acceptable salt thereof.

6. The method of claim 5 wherein the acyl radical is attached to the epsilon amino group of a lysine residue of the GLP-1 peptide via a linker, wherein the linker preferably comprises at least one OEG radical (OEG is 8-amino-3,6-dioxaoctanic acid), at least one Trx radical (Trx is tranexamic acid, or trans-4-(aminomethyl)cyclohexanecarboxylic acid): 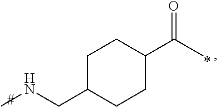
and/or at least one Glu (glutamine) radical.

7. The method of claim 1, wherein the GLP-1 compound is selected from:
N-epsilon26-((S)-4-carboxy-4-hexadecanoylaminobutyryl)[Arg34]GLP-1-(7-37): 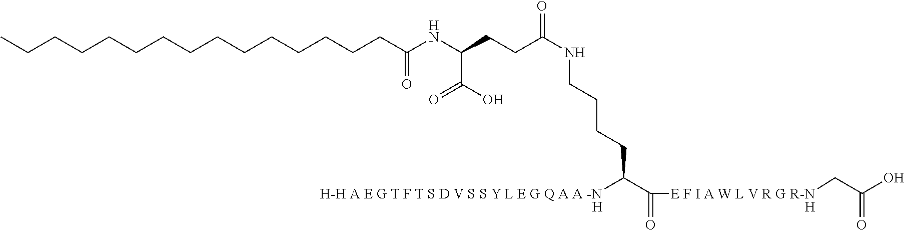
(compound G1);
N-epsilon37-[2-(2-{2-[2-(2-{2-[(S)-4-carboxy-4-({trans-4-[(19-carboxynonadecanoyl-amino)methyl]cyclohexanecarbonyl}amino)butyrylamino]ethoxy}ethoxy)acetylamino]-ethoxy}ethoxy)acetyl][DesaminoHis7,Glu22,Arg26,Arg34,Lys37]GLP-1-(7-37): 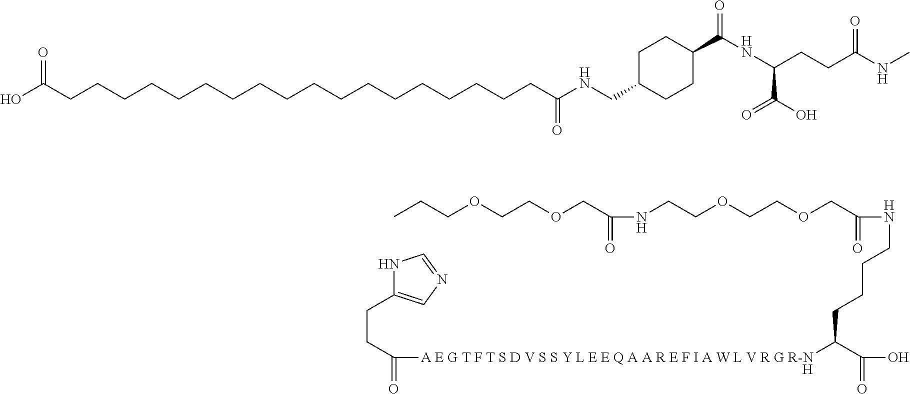
(compound G2);
N-epsilon26-[2-(2-{2-[2-(2-{2-[(S)-4-carboxy-4-(17-carboxyheptadecanoylamino)butyrylamino]ethoxy}ethoxy)acetylamino]-ethoxy}ethoxy)acetyl][Aib8,Arg34] GLP-1-(7-37): 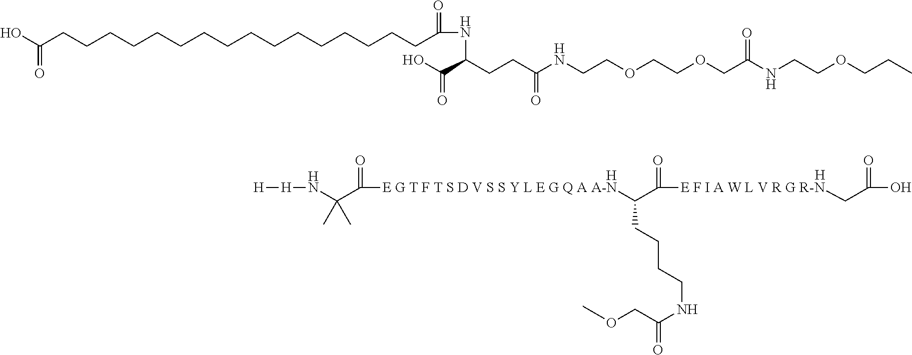
(compound G3);
N-epsilon37-[2-(2-{2-[2-(2-{2-[(S)-4-carboxy-4-(15-carboxypentadecanoylamino)-butyrylamino]ethoxy}ethoxy)acetylamino]ethoxy}ethoxy)acetyl][Aib8,22,35,Lys37]GLP-1-(7-37): 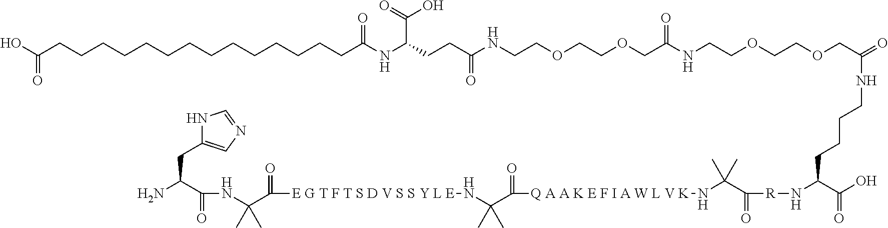
(compound G4);
and their pharmaceutically acceptable salts, amides, alkyls, or esters.

8. The method of any one of claim 1, wherein the FGF21 compound comprises the amino acid sequence of SEQ ID NO:1 or is an analogue thereof having a maximum of 30 amino acid substitutions, deletions, and/or additions.

9. The method of claim 1, wherein the FGF21 compound is an FGF21 derivative comprising an albumin binding moiety.

10. The method of claim 9, wherein the albumin binding moiety comprises at least one, preferably at least two, more preferably two, free carboxylic acid groups, or a pharmaceutically acceptable salt thereof.

11. The method of claim 9, wherein the albumin binding moiety comprises an acyl radical, such as acyl of fatty acids or dicarboxylic acids, for example 17-carboxy-heptadecanoyl- and 19-carboxynonadecanoyl-, the acyl radical preferably comprising a total of from 12 to 24 carbon atoms, such as C12, C14, C16, C18, C20, C22, or C24, most preferably C18, or C20; or a pharmaceutically acceptable salt thereof.

12. The method of claim 11, wherein the acyl radical is attached to the amino group of the N-terminal amino acid residue of the FGF21 peptide, e.g., to the amino group of -1M, or to the thiol group of an internal cysteine residue of the FGF21 peptide, e.g. to the thiol group of 71C, via a linker, wherein the linker preferably comprises at least one OEG radical (OEG is 8-amino-3,6-dioxaoctanic acid), and/or at least one Glu (glutamine) radical.

13. The method of claim 1, wherein the FGF21 compound is selected from the group consisting of:
the polypeptide having SEQ ID NO:1 (human FGF21);
the polypeptide having SEQ ID NO: 1 with an added N-terminal Met (Met-FGF21_human, compound F1);
S-71-({2-[2-(2-{2-[2-(2-{2-[(S)-4-carboxy-4-(19-carboxynonadecanoylamino)butyrylamino]ethoxy}-ethoxy)acetylamino]ethoxy}ethoxy)acetylamino]ethylcarbamoyl methyl)[Cys71]Met-FGF21 (compound F2); and
N-alpha1-[2-(2-{2-[2-(2-{2-[(S)-4-carboxy-4-(17-carboxyheptadecanoylamino)butyrylamino]-ethoxy}ethoxy)acetylamino]ethoxy}ethoxy)acetyl][Arg56, Arg59, Arg69, Arg122]-Met-FGF21 (compound F3);
and their pharmaceutically acceptable salts, amides, alkyls, or esters.

14. The method of claim 1, wherein the compounds are administered simultaneously, and/or sequentially.

15. (canceled)


SEQUENCE LISTING (4 sequences)

SEQ ID NO: 1 - protein, 181 aa, Homo sapiens
  mat_peptide (1)..(181)
  DISULFID (75)..(93)
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 2 - protein, 28 aa, Homo sapiens
  SIGNAL (1)..(28)
MDSDETGFEHSGLWVSVLAGLLLGACQA

SEQ ID NO: 3 - protein, 31 aa, Homo sapiens
  mat_peptide (1)..(31)
HAEGTFTSDVSSYLEGQAAKEFIAWLVKGRG

SEQ ID NO: 4 - protein, 39 aa, Heloderma suspectum
  mat_peptide (1)..(39)
HGEGTFTSDLSKQMEEEAVRLFIEWLKNGGPSSGAPPPS